(19) United States
(12) Patent Application Publication
(10) Pub. No.: US 2009/0074690 A1
(43) Pub. Date: Mar. 19, 2009

(54) COSMETIC COMPOSITIONS COMPRISING AT LEAST ONE CATIONIC COPOLYMER AND AT LEAST ONE TRIGLYCERIDE AND METHODS OF USE THEREOF

(76) Inventors: Olga BIGANSKA, Asnieres sur Seine (FR); Pascale Lazzeri-Vigouroux, Puyricard (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/210,773

(22) Filed: Sep. 15, 2008

Related U.S. Application Data
(60) Provisional application No. 60/960,232, filed on Sep. 21, 2007.

(30) Foreign Application Priority Data
Sep. 14, 2007 (FR) 0757583

(51) Int. Cl. A61K 8/72; A61Q 5/02; A61Q 5/04; A61Q 5/08; A61Q 5/10; A61Q 5/12; A61K 8/81; A61Q 1/14; A61Q 19/10

(52) U.S. Cl. 424/62; 424/70.11; 424/70.2; 8/405; 510/119; 510/136; 510/135; 510/158

(57) ABSTRACT

The present disclosure relates to novel cosmetic compositions comprising, in a cosmetically acceptable medium
(i) at least one cationic polymer, produced by polymerization of a monomer mixture comprising:
   a) at least one vinyl monomer substituted with at least one amino group,
   b) at least one hydrophobic nonionic vinyl monomer, chosen from formulae (I) and (II):

CH₂═C(X)Z, (I)

CH₂═CH—OC(O)R; (II)
wherein:
X is chosen from a hydrogen atom and a methyl group;
Z is chosen from the groups —C(O)OR¹, —C(O)NH₂, —C(O)NHR¹, —C(O)N(R¹)₂, —C₆H₅, —C₆H₄R¹, —C₆H₄OR¹, —C₆H₄Cl, —CN, —NHC(O)CH₃, —NHC(O)H, N-(2-pyrrolidonyl), N-caprolactamyl, —C(O)NHC(CH₃)₃, —C(O)NHCH₂CH₂—NH—CH₂CH₂-urea, —Si(R)₃, —C(O)O(CH₂)ₓSi(R)₃, —C(O)NH(CH₂)ₓSi(R)₃, and —(CH₂)ₓSi(R)₃;
x is an integer ranging from 1 to 6;
each R is independently chosen from ^{a}C₁-C₃₀ alkyl group;
each R¹ is independently chosen from ^{a}C₁-C₃₀ alkyl group, a C₂-C₃₀ hydroxyalkyl group, and a C₁-C₃₀ haloalkyl group, and
   c) at least one associative vinyl monomer,
   e) at least one hydroxylated nonionic vinyl monomer,
(ii) at least one saturated or unsaturated C₁₂-C₃₀ fatty acid triglyceride, the content of C18:2 acids of the triglyceride being less than 50% by weight relative to the total weight of the fatty acids of the triglyceride, and
(iii) at least one non-silicone cationic polymer other than the at least one cationic polymer (i).

These compositions may be used for washing and/or conditioning keratin materials such as the hair or the skin.

This application claims benefit of U.S. Provisional Application No. 60/960,232, filed Sep. 21, 2007, the contents of which are incorporated herein by reference. This application also claims benefit of priority under 35 U.S.C. § 119 to French Patent Application No. FR 0757583, filed Sep. 14, 2007, the contents of which are also incorporated herein by reference.

Disclosed herein are novel cosmetic compositions comprising, in a cosmetically acceptable medium, at least one triglyceride and at least one cationic copolymer.

It is well known that hair which has been sensitized (i.e., damaged and/or embrittled) to varying degrees by the action of atmospheric agents or the action of mechanical or chemical treatments, such as dyes, bleaches and/or permanent-waving, is often difficult to disentangle and to style, and lacks softness.

It is known to use conditioners, such as cationic polymers or silicones, in compositions for washing or caring for keratin material such as the hair, in order to disentangle the hair and to give it softness and flexibility. However, these polymers or silicones may have the disadvantage of lankness of the hairstyle (lack of lightness of the hair) and lack of smoothness (hair not uniform from the root to the end) on dried hair.

In addition, the use of cationic polymers for this purpose has other drawbacks. On account of their high affinity for the hair, some of these polymers become deposited thereon to a large extent during repeated use, and lead to adverse effects such as an unpleasant, laden feel, stiffening of the hair and interfiber adhesion which has an effect on styling. These drawbacks are accentuated with fine hair, which lacks liveliness and body.

It is also known to use as conditioning agent, oils such as plant or animal oils or fatty acid esters. However, keratin materials treated with these compositions usually have an unacceptable greasy feel.

To thicken and stabilize compositions comprising insoluble conditioning agents, stabilizers such as crosslinked acrylic polymers of the type CARBOPOL 980 are frequently used. However, these stabilizers have the drawback of reducing the cosmetic performance of shampoos, for example, by making the hair more laden and more coarse. Furthermore, the compositions are generally not stable over time, especially at 45° C.

For instance, International Patent Application Publication No. WO 2004/024779 describes detergent compositions comprising a cationic copolymer as an agent for stabilizing or suspending water-insoluble ingredients such as silicones or fatty substances. The cosmetic properties obtained with these compositions are still not sufficiently satisfactory.

The present inventors have discovered that the combination of at least one cationic copolymer and at least one triglyceride makes it possible to overcome these drawbacks.

Specifically, it has been found that the combination of at least one cationic copolymer and at least one triglyceride makes it possible to obtain on keratin materials, such as the hair, improved deposition of the triglyceride, which leads to very good cosmetic properties, for example, sheen, while also affording lightness, ease of disentangling, smoothness, softness, a uniform and natural feel, and suppleness without any sensation of a laden feel.

Moreover, the compositions according to the present disclosure are stable and have a very pleasant aesthetic visual appearance and texture.

The working properties, for instance appearance, consistency, foam abundance, and elimination of foam are very satisfactory.

These compositions are stable and have a texture that changes little or not at all over time at room temperature (about 25° C.) and at 45° C. These compositions afford better distribution of the product during application and better foaming properties such as improved initiation of foam.

Moreover, the compositions disclosed herein, when applied to the skin, such as in the form of a bubble bath or a shower gel, improve the softness of the skin.

Thus, one aspect of the present disclosure are novel cosmetic compositions comprising, in a cosmetically acceptable medium, (i) at least one cationic polymer, produced by polymerization of a monomer mixture comprising:
a) at least one vinyl monomer substituted with at least one amino group,
b) at least one hydrophobic nonionic vinyl monomer, chosen from formulae (I) and (II):

CH₂═C(X)Z, (I)

CH₂═CH—OC(O)R; (II)

wherein:

X is chosen from a hydrogen atom and a methyl group;

Z is chosen from the groups —C(O)OR¹, —C(O)NH₂, —C(O)NHR¹, —C(O)N(R¹)₂, —C₆H₅, —C₆H₄R¹, —C₆H₄OR¹, —C₆H₄Cl, —CN, —NHC(O)CH₃, —NHC(O)H, N-(2-pyrrolidonyl), N-caprolactamyl, —C(O)NHC(CH₃)₃, —C(O)NHCH₂CH₂—NH—CH₂CH₂-urea, —Si(R)₃, —C(O)O(CH₂)ₓSi(R)₃, —C(O)NH(CH₂)ₓSi(R)₃, and —(CH₂)ₓSi(R)₃;

x is an integer ranging from 1 to 6;

each R is independently chosen from a C₁-C₃₀ alkyl group;

each R¹ is independently chosen from a C₁-C₃₀ alkyl group, a C₂-C₃₀ hydroxyalkyl group, and a C₁-C₃₀ haloalkyl group,
c) at least one associative vinyl monomer, and
e) at least one hydroxylated nonionic vinyl monomer,
(ii) at least one saturated or unsaturated C₁₂-C₃₀ fatty acid triglyceride, the content of C18:2 acids of the triglyceride being less than 50% by weight relative to the total weight of the fatty acids of the triglyceride, and
(iii) at least one non-silicone cationic polymer other than the at least one cationic polymer (i).

Another aspect of the present disclosure is the use of a composition disclosed herein for giving the hair at least one property chosen from lightness, softness, smooth feel, and/or suppleness.

As used herein, “keratin materials” means the hair, the eyelashes, the eyebrows, the skin, the nails, mucous membranes, or the scalp, such as the hair.

As used herein, “non-silicone cationic polymers” means that the cationic polymers do not comprise any Si—O bonds.

Another aspect of the present disclosure relates to the use of a cationic copolymer as disclosed herein, or for the manufacture of a cosmetic composition comprising at least one triglyceride as defined below.

As used herein, “saturated or unsaturated C₁₂-C₃₀ fatty acid triglycerides” means triesters of glycerol and of saturated or unsaturated fatty acids comprising from 12 to 30 carbon atoms. These triglycerides are not oxyalkylenated.

The triglycerides according to the present disclosure are generally liquid at 25° C., and have a melting point, for example, of less than 35° C.

As used herein, “C18:2 acids” means carboxylic acids comprising 18 carbon atoms and two carbon-carbon double bonds. The most common of these acids is linoleic acid.

One of the essential characteristics of the present disclosure is the presence of at least one cationic polymer that is obtained by polymerization of a monomer mixture comprising a) at least one vinyl monomer substituted with at least one amino group, b) at least one hydrophobic nonionic vinyl monomer, c) at least one associative vinyl monomer, and e) at least one hydroxylated nonionic vinyl monomer.

In at least one embodiment of the present disclosure, the monomers constituting the at least one cationic copolymer also comprise at least one semi-hydrophobic vinyl surfactant monomer d). The monomers a) to e) are different from one another.

In at least one embodiment, the at least one cationic polymer (i) is a thickening polymer.

As used herein, “thickening polymer” means a polymer which, when introduced at 1% by weight into an aqueous or aqueous-alcoholic solution comprising 30% by weight of ethanol, and at pH 7, makes it possible to achieve a viscosity of at least 100 cps at 25° C., and at a shear rate of 1 s⁻¹. This viscosity may be measured using a viscometer with cone-plate geometry, for example, a Haake RS 600 rheometer. In at least one embodiment, these polymers make it possible to increase the viscosity of the compositions in which they are present by at least 50 cps at 25° C. and at 1 s⁻¹.

The at least one cationic polymer (i) used in the composition according to the present disclosure, and the process for manufacturing them, are described, for instance, in International Patent Application No. WO 2004/024779.

As used herein, “vinyl monomer” means a monomer comprising at least one group chosen from R₀CH═C(Ro)—, wherein each Ro is independently chosen from a hydrogen atom, C₁-C₃₀ alkyl, —C(O)OH, C(O)OR₀′, —O—C(O)OR₀′, —C(O)NHR₀′, —C(O)NHR₀′, and C(O)NR₀′R₀″, wherein R₀′ and R₀″, which may be identical or different, are chosen from C₁-C₃₀ alkyl groups. Such vinyl monomers include, for example, (meth)acrylates and (meth)acrylamides.

According to the present disclosure, the monomer mixture for preparing the at least one cationic polymer (i) used in the composition disclosed herein comprises at least one vinyl monomer substituted with at least one amino group.

According to the present disclosure, the at least one vinyl monomer substituted with at least one amino group a) that may be used for the preparation of the at least one cationic polymer (i) disclosed herein, is chosen from basic, polymerizable ethylenically unsaturated monomers. The at least one amine group may be derived from monoamino, diamino and polyamino alkyl groups, or from heteroaromatic groups comprising a nitrogen atom. The at least one amine group may be chosen from a primary, secondary, and tertiary amine. These monomers may be used in the form of amine or in the form of salt.

In one embodiment, the at least one vinyl monomer substituted with at least one amine group a) is chosen from:

mono(C₁-C₄)alkylamino(C₁-C₈)alkyl (meth)acrylates,

di(C₁-C₄)alkylamino(C₁-C₈)alkyl (meth)acrylates, such as di(C₁-C₄)alkylamino(C₁-C₆)alkyl (meth)acrylates,

mono(C₁-C₄)alkylamino(C₁-C₈)alkyl(meth)acrylamides,

di(C₁-C₄)alkylamino(C₁-C₈)alkyl(meth)acrylamides,

(meth)acrylamides with at least one heterocyclic group comprising a nitrogen atom,

(meth)acrylates with at least one heterocyclic group comprising a nitrogen atom,

nitrogenous heterocycles comprising at least one vinyl group,

and mixtures thereof.

Non-limiting examples of the at least one vinyl monomer substituted with at least one amino group a) that may be mentioned include:

mono- or di(C₁-C₄ alkyl)amino(C₁-C₄alkyl) (meth)acrylates, such as 2-(N,N-dimethylamino)ethyl (meth)acrylate, 3-(N,N-dimethylamino)propyl (meth)acrylate, 4-(N,N-dimethylamino)butyl (meth)acrylate, (N,N-dimethylamino)-t-butyl (meth)acrylate, 2-(N,N-diethylamino)ethyl (meth)acrylate, 3-(N,N-diethylamino)propyl (meth)acrylate, 4-(N,N-diethylamino)butyl (meth)acrylate, 2-(N,N-dipropylamino)ethyl (meth)acrylate, 3-(N,N-dipropylamino)propyl (meth)acrylate, and 4-(N,N-dipropylamino)butyl (meth)acrylate;

mono- or di(C₁-C₄ alkyl)amino(C₁-C₄ alkyl)(meth)acrylamides, such as N′-(2-N,N-dimethylamino)ethyl(meth)acrylamide and N′-(3-N,N-dimethylamino)propylacrylamide;

(meth)acrylamides or (meth)acrylates with a heterocyclic group comprising a nitrogen atom, such as N-(2-pyridyl)acrylamide, N-(2-imidazolyl)methacrylamide, 2-(4-morpholinyl)ethyl methacrylate, 2-(4-morpholinyl)ethyl acrylate, N-(4-morpholinyl)methacrylamide, and N-(4-morpholinyl)acrylamide; and

nitrogenous heterocycles comprising at least one vinyl group, such as 2-vinylpyridine and 4-vinylpyridine.

When the at least one monomer is in the form of a salt, it may be a mineral salt, such as a hydrochloride, sulfate, or phosphate salt; or an organic acid salt, such as an acetate, maleate, or fumarate salt.

In at least one embodiment, the at least one vinyl monomer substituted with at least one amino group a) is chosen from, by way of non-limiting example:
3-(N,N-dimethylamino)propyl (meth)acrylate,
N′-(3-N,N-dimethylamino)propyl(meth)acrylamide,
2-(N,N-dimethylamino)ethyl (meth)acrylate,
2-(N,N-diethylamino)ethyl (meth)acrylate,
2-(tert-butylamino)ethyl (meth)acrylate,
2-(N,N-dimethylamino)propyl(meth)acrylamide, and
2-(N,N-dimethylamino)neopentyl acrylate.

The at least one vinyl monomer substituted with at least one amino group a) is present in an amount ranging from 10% to 70% by weight, for example, from 20% to 60% by weight, and further for example, from 30% to 40% by weight, relative to the total weight of the monomer mixture.

According to the present disclosure, the monomer mixture for preparing the at least one cationic polymer (i) disclosed herein also comprises at least one hydrophobic nonionic vinyl monomer b).

The at least one hydrophobic nonionic vinyl monomer b) for the preparation of the at least one cationic polymer (i) disclosed herein is, in at least one embodiment, chosen from compounds of formulae (I) and (II):

CH₂═C(X)Z, (I)

CH₂═CH—OC(O)R; (II)

wherein:
X is chosen from a hydrogen atom and a methyl group;
Z is chosen from the groups —C(O)OR¹, —C(O)NH₂, —C(O)NHR¹, —C(O)N(R¹)₂, —C₆H₅, —C₆H₄R¹, —C₆H₄OR¹, —C₆H₄Cl, —CN, —NHC(O)CH₃, —NHC(O)H, N-(2-pyrrolidonyl), N-caprolactamyl, —C(O)NHC(CH₃)₃, —C(O)NHCH₂CH₂—NH—CH₂CH₂-urea, —Si(R)₃, —C(O)O(CH₂)ₓSi(R)₃, —C(O)NH(CH₂)ₓSi(R)₃, and —(CH₂)ₓSi(R)₃;
x is an integer ranging from 1 to 6;
each R is independently chosen from a C₁-C₃₀ alkyl group;
each R¹ is independently chosen from a C₁-C₃₀ alkyl group, a C₂-C₃₀ hydroxyalkyl group, and a C₁-C₃₀ haloalkyl group.

Mention may be made in a non-limiting manner of C₁-C₃₀ alkyl (meth)acrylates; (C₁-C₃₀ alkyl)(meth)acrylamides; styrene, substituted styrenes, such as vinyltoluene (or 2-methylstyrene), butylstyrene, isopropylstyrene, and para-chlorostyrene; vinyl esters, such as vinyl acetate, vinyl butyrate, vinyl caprylate, vinyl pidolate, and vinyl neodecanoate; unsaturated nitriles, for example, (meth)acrylonitrile and acrylonitrile; and unsaturated silanes, for instance trimethylvinylsilane, dimethylethylvinylsilane, allyldimethylphenylsilane, allyltrimethylsilane, 3-acrylamidopropyltrimethylsilane, and 3-trimethylsilylpropyl methacrylate.

In one embodiment, the at least one hydrophobic nonionic vinyl monomer b) is chosen from C₁-C₃₀ alkyl acrylates, C₁-C₃₀ alkyl methacrylates, and mixtures thereof, such as ethyl acrylate, methyl methacrylate, and 3,3,5-trimethylcyclohexyl methacrylate.

The at least one hydrophobic nonionic vinyl monomer b) is present in an amount ranging from 20% to 80% by weight, for example from 20% to 70% by weight, and further for example from 50% to 65% by weight, relative to the total weight of the monomer mixture.

The at least one associative vinyl monomer c) that may be used for the preparation of the at least one cationic polymer (i) disclosed herein may be chosen, by way of non-limiting example, from compounds having an ethylenically unsaturated end (i)′ for addition polymerization with other monomers of the system; a polyoxyalkylene central portion (ii)′ for giving the polymers selective hydrophilic properties, and a hydrophobic end (iii)′ for giving the polymers selective hydrophobic properties.

The ethylenically unsaturated end (i)′ of the at least one associative vinyl monomer c) is for example, derived from an α,β-ethylenically unsaturated monocarboxylic or dicarboxylic acid or anhydride, such as a C₃ or C₄ monocarboxylic or dicarboxylic acid or anhydride. Alternatively, the end (i)′ of the at least one associative monomer c) may be derived from an allyl ether or a vinyl ether; from a nonionic urethane monomer substituted with a vinyl group, as described in U.S. Pat. No. Re. 33,156 or in U.S. Pat. No. 5,294,692; or a product of reaction of urea substituted with a vinyl group, such as described in U.S. Pat. No. 5,011,978.

The central portion (ii)′ of the at least one associative vinyl monomer c) is, for instance, a polyoxyalkylene segment comprising 5 to 250 C₂-C₇ alkylene oxide units, for example 10 to 120 C₂-C₇ alkylene oxide units, and further for example 15 to 60 C₂-C₇ alkylene oxide units. In one embodiment, the central portions (ii)′ are, for example, polyoxyethylene, polyoxypropylene, and polyoxybutylene segments comprising 5 to 150, such as from 10 to 100, and further for instance 15 to 60 ethylene oxide, propylene oxide, or butylene oxide units, and random or non-random blocks of ethylene oxide, propylene oxide, or butylene oxide units. In one embodiment, the central portions (ii)′ are polyoxyethylene segments.

The hydrophobic end (iii)′ of the at least one associative monomer c) is, for instance, a hydrocarbon-based fragment chosen from linear alkyls, C₂-C₄₀ alkyls substituted with an aryl group, phenyls substituted with a C₂-C₄₀ alkyl group, branched alkyls, alicyclic groups, and complex esters.

As used herein, “complex ester” means any ester other than a simple ester.

As used herein, “simple ester” means any ester of an unsubstituted, linear or branched saturated C₁-C₃₀ aliphatic alcohol.

Non-limiting examples of hydrophobic ends (iii)′ of the at least one monomer c) include C₈-C₄₀ linear and branched alkyl groups, such as capryl (C₈₋), isooctyl (branched C₈), decyl (C₁₀), lauryl (C₁₂), myristyl (C₁₄), cetyl (C₁₆), cetearyl (C₁₆-C₁₈), stearyl (C₁₈), isostearyl (branched C₁₈), arachidyl (C₂₀), behenyl (C₂₂), lignoceryl (C₂₄), cerotyl (C₂₆), montanyl (C₂₈), melissyl (C₃₀), and lacceryl (C₃₂) groups.

Non-limiting examples of C₈-C₄₀ linear and branched alkyl groups derived from a natural source include alkyl groups derived from hydrogenated groundnut oil, soybean oil and canola oil (predominantly C₁₈), C₁₆-C₁₈ hydrogenated tallow oil; and C₁₀-C₃₀ hydrogenated terpenols, such as hydrogenated geraniol (branched C₁₀), hydrogenated farnesol (branched C₁₅), and hydrogenated phytol (branched C₂₀).

Non-limiting examples of phenyls substituted with a C₂-C₄₀ alkyl include octylphenyl, nonylphenyl, decylphenyl, dodecylphenyl, hexadecylphenyl, octadecylphenyl, isooctylphenyl, and sec-butylphenyl.

C₈-C₄₀ alicyclic groups may be, for example, groups derived from sterols of animal origin, such as cholesterol, lanosterol and 7-dehydrocholesterol; or derivatives of plant origin, such as phytosterol, stigmasterol, or campesterol; or derivatives obtained from microorganisms, such as ergosterol or mycrosterol. Other useful C₈-C₄₀ alicyclics include, but are not limited to, cyclooctyl, cyclododecyl, adamantyl, and decahydronaphthyl, and groups derived from natural C₈-C₄₀ alicyclics compounds such as pinene, hydrogenated retinol, camphor, and isobornyl alcohol.

The C₂-C₄₀ alkyl groups substituted with an aryl group may be chosen from, for example, 2-phenylethyl, 2,4-diphenybutyl, 2,4,6-triphenylhexyl, 4-phenylbutyl, 2-methyl-2-phenylethyl, and 2,4,6-tris(1′-phenylethyl)phenyl.

Suitable C₈-C₄₀ complex esters that may be used herein include, but are not limited to, hydrogenated castor oil (mainly 12-hydroxystearic acid triglyceride); 1,2-diacyl glycerols, such as 1,2-distearyl glycerol, 1,2-dipalmitol glycerol, and 1,2-dimyristyl glycerol; di-, tri-, or polyesters of sugars, such as 3,4,6-tristearyl glucose or 2,3-dilauryl fructose; and sorbitan esters, such as those described in U.S. Pat. No. 4,600,761.

The at least one associative vinyl monomer c) that may be used according to the present disclosure may be prepared via any method known in the prior art, for example, the methods described in U.S. Pat. Nos. 4,421,902; 4,384,096; 4,514,552; 4,600,761; 4,616,074; 5,294,692; 5,292,843; 5,770,760 and 5,412,142.

In at least one embodiment, the at least one associative vinyl monomer c) that may be used according to the present disclosure is chosen from compounds of formula (III):

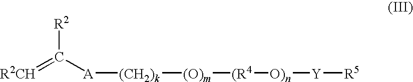

wherein:
each R² is independently chosen from a hydrogen atom a methyl group,
a —C(O)OH group, and a —C(O)OR³ group;
R³ is a C₁-C₃₀ alkyl;
A is chosen from —CH₂C(O)O—, —C(O)O—, —O—, CH₂O, —NHC(O)NH—, —C(O)NH—, —Ar—(CE₂)_{z}—NHC(O)O—, —Ar—(CE₂)_{z}-NHC(O)NH—, and —CH₂CH₂—NHC(O)—;
Ar is a divalent aryl group;
E is chosen from a hydrogen atom and a methyl group;
z is an integer ranging from 0 to 1;
k is an integer ranging from 0 to 30;
m is an integer ranging from 0 to 1, with the proviso that when k is 0, then m is 0, and when
k is an integer ranging from 1 to 30, then m is 1;
(R⁴—O)ₙ is a polyoxyalkylene, which is a homopolymer, a random copolymer or a block copolymer, with C₂-C₄ oxyalkylene units;
R⁴ is chosen from C₂H₄, C₃H₆, C₄H₈, and mixtures thereof;
n is an integer ranging from 5 to 250;
Y is chosen from —R⁴⁰—, —R⁴NH—, —C(O)—, —C(O)NH—, R⁴NHC(O)NH—, and —C(O)NHC(O)—;
R⁵ is chosen from substituted and unsubstituted alkyls such as linear C₈-C₄₀ alkyl groups, branched C₈-C₄₀ alkyl groups, C₈-C₄₀ alicyclic groups, phenyls substituted with a C₂-C₄₀ alkyl group, C₂-C₄₀ alkyl groups substituted with an aryl group, and C₈-C₈₀ complex esters, wherein
the alkyl group R⁵ optionally comprises at least one substituent chosen from hydroxyl, alkoxy, and halo groups.

In one embodiment, the at least one associative vinyl monomer c) is chosen from polyethoxylated cetyl (meth)acrylates, polyethoxylated cetearyl (meth)acrylates, polyethoxylated stearyl (meth)acrylates, polyethoxylated arachidyl (meth)acrylates, polyethoxylated behenyl (meth)acrylates, polyethoxylated lauryl (meth)acrylates, polyethoxylated cerotyl (meth)acrylates, polyethoxylated montanyl (meth)acrylates, polyethoxylated melissyl (meth)acrylates, polyethoxylated lacceryl (meth)acrylates, polyethoxylated 2,4,6-tris(1′-phenylethyl)phenyl (meth)acrylates, polyethoxylated hydrogenated castor oil (meth)acrylates, polyethoxylated canola (meth)acrylates, polyethoxylated cholesteryl (meth)acrylates, and mixtures thereof, wherein the polyethoxylated portion of the monomer comprises from 5 to 100 ethylene oxide units, for example from 10 to 80 ethylene oxide units, and further for example from 15 to 60 ethylene oxide units.

In at least one embodiment, the at least one associative vinyl monomer c) is chosen from polyethoxylated cetyl methacrylates, polyethoxylated cetearyl methacrylates, polyethoxylated stearyl (meth)acrylates, polyethoxylated arachidyl (meth)acrylates, polyethoxylated behenyl (meth)acrylates and polyethoxylated lauryl (meth)acrylates, wherein the polyethoxylated portion of the monomer comprises from 10 to 80 ethylene oxide units, such as from 15 to 60-ethylene oxide units, and further such as from 20 to 40 ethylene oxide units.

According to at least one embodiment of the present disclosure, the at least one associative vinyl monomer c) is present in an amount ranging from 0.001% to 25% by weight, for example from 0.01% to 15% by weight, and further for example from 0.1% to 10% by weight, relative to the total weight of the monomer mixture.

The at least one semi-hydrophobic vinyl surfactant monomer d) optionally present in the monomer mixture can moderate the associative properties of the cationic associative polymers that comprise them, thus may produce aqueous gels having a very good texture and very good rheological properties.

As used herein, “semi-hydrophobic vinyl surfactant monomer” means a monomer with a structure similar to that of an associative monomer, but which has a substantially non-hydrophobic end and thus does not give the polymers associative properties.

The associative property of a polymer is linked to the property in a given medium, of the molecules of the said polymer to associate with each other, or to associate with molecules of a co-agent, such as a surfactant, which is reflected in a certain concentration range by an increase in the viscosity of the medium.

The at least one semi-hydrophobic vinyl surfactant monomer d) is generally a compound comprising two parts:

A. an unsaturated end group to allow addition polymerization with the other monomers of the reaction mixture, and

B. a polyoxyalkylene group to attenuate the associations between the hydrophobic groups of the polymer or the hydrophobic groups of the other materials that may be present in the composition comprising the polymer.

The end A providing the vinyl or ethylenic unsaturation for the addition polymerization may be derived, for example, from an α,β-ethylenically unsaturated monocarboxylic or dicarboxylic acid or anhydride, such as a C₃-C₄ monocarboxylic or dicarboxylic acid, or an anhydride of this acid. In one embodiment, the end A may be derived from an allylic ether, a vinyl ether, or a nonionic unsaturated urethane.

The polymerizable unsaturated end A may also be derived from a C₈-C₃₀ unsaturated fatty acid comprising at least one free carboxyl functional group. This C₈-C₃₀ group forms part of the unsaturated end A and is different from the pendent hydrophobic groups of the at least one associative monomer c), which are separated from the unsaturated end of the associative monomer by a hydrophilic spacer group.

The polyoxyalkylene portion B comprises a long-chain polyoxyalkylene segment, which is similar to the hydrophilic portion of the at least one associative monomer c). Non-limiting examples of polyoxyalkylene portions B include C₂-C₄ polyoxyethylene, polyoxypropylene, and polyoxybutylene units comprising from 5 to 250 oxyalkylene units, such as from 10 to 100 oxyalkylene units. When the at least one semi-hydrophobic vinyl surfactant monomer d) comprises more than one type of oxyalkylene unit, these units may be distributed randomly, non-randomly, or in blocks.

In at least one embodiment, the at least one semi-hydrophobic vinyl surfactant monomer d) is chosen from the compounds of formula (IV) and (V):

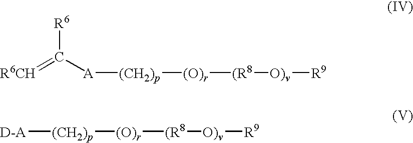

wherein:
each R⁶ is independently chosen from a hydrogen atom, a C₁-C₃₀ alkyl, —C(O)OH, and C(O)OR⁷;
R⁷ is a C₁-C₃₀ alkyl;
A is chosen from —CH₂C(O)O—, —C(O)O—, —O—, —CH₂O, —NHC(O)NH—, —C(O)NH—, —Ar—(CE₂)_{z}—NHC(O)O—, —Ar—(CE₂)_{z}-NHC(O)NH—, and —CH₂CH₂NHC(O)—;
Ar is a divalent aryl group;
E is chosen from a hydrogen atom and a methyl group;
z is an integer ranging from 0 to 1;
p is an integer ranging from 0 to 30;
r is an integer ranging from 0 to 1, with the provisos that when p is 0, then r is 0, and when
p is an integer ranging from 1 to 30, then r is 1;

(R₈—O)ᵥ is a polyoxyalkylene which is a homopolymer, a random copolymer, or a block copolymer with C₂-C₄ oxyalkylene units, wherein R⁸ is chosen from C₂H₄, C₃H₆, C₄H₈, and mixtures thereof, and v is an integer ranging from 5 to 250;

R⁹ is chosen from a hydrogen atom and a C₁-C₄ alkyl; and
D is chosen from C₈-C₃₀ alkenyl groups optionally substituted with a carboxyl group.

In at least one embodiment of the present disclosure, the monomer mixture comprises at least one semi-hydrophobic vinyl surfactant monomer d) chosen from one of the following formulae:

CH₂═CH—O(CH₂)ₐO(C₃H₆O)_{b}(C₂H₄O)_{c}H, and

CH₂═CHCH₂O(C₃H₆O)_{d}(C₂H₄O)ₑH;

wherein:
a is an integer ranging from 2 to 4;
b is an integer ranging from 1 to 10;
c is an integer ranging from 5 to 50;
d is an integer ranging from 1 to 10; and
e is an integer ranging from 5 to 50.

Non-limiting examples of the at least one semi-hydrophobic vinyl surfactant monomer d) include the polymerizable emulsifiers sold under the references EMULOSGEN® RH 09, R208, R307, RAL109, RAL208, and RAL307 by the company Clariant; BX-AA-E5P5 sold by the company Bimax; and MAXEMUL® 5010 and 5011 sold by the company Uniqema. In at least one embodiment, mention may be made, in a non-limiting manner, of the monomers sold under the name Emulsogen® R208, R307, and RAL 307.

According to the manufacturers:

EMULSOGEN® R109 is a random ethoxylated/propoxylated 1,4-butanediol vinyl ether having the empirical formula:

CH₂═CH—O(CH₂)₄O(C₃H₆O)₄(C₂H₄O)₁₀H;

EMULSOGEN® R208 is a random ethoxylated/propoxylated 1,4-butanediol vinyl ether having the empirical formula:

CH₂═CH—O(CH₂)₄O(C₃H₆O)₄(C₂H₄O)₂₀H;

EMULSOGEN® R307 is a random ethoxylated/propoxylated 1,4-butanediol vinyl ether having the empirical formula:

CH₂═CH—O(CH₂)₄O(C₃H₆O)₄(C₂H₄O)₃₀H;

EMULSOGEN® RAL 109 is a random ethoxylated/propoxylated allylic ether having the empirical formula:

CH₂═CHCH₂—O(C₃H₆O)₄(C₂H₄O)₁₀H;

EMULSOGEN® RAL 208 is a random ethoxylated/propoxylated allylic ether having the empirical formula:

CH₂═CHCH₂—O(C₃H₆O)₄(C₂H₄O)₂₀H;

EMULSOGEN® RAL 307 is a random ethoxylated/propoxylated allylic ether having the empirical formula:

CH₂═CHCH₂—O(C₃H₆O)₄(C₂H₄O)₃₀H;

MAXEMUL® 5010 is a hydrophobic carboxylated C₁₂-C₁₅ alkenyl, ethoxylated with 24 ethylene oxide units,
MAXEMUL® 5011 is a hydrophobic carboxylated C₁₂-C₁₅ alkenyl, ethoxylated with 34 ethylene oxide units; and
BX-AA-E5P5 is a random ethoxylated/propoxylated allylic ether having the empirical formula:

CH₂═CHCH₂—O(C₃H₆O)₅(C₂H₄O)₅H.

The amount of the at least one semi-hydrophobic vinyl surfactant monomer d) used in the preparation of the at least one cationic polymer (i) in the compositions disclosed herein may vary widely and also depends on the final rheological properties desired for the polymer.

When present, the at least one semi-hydrophobic vinyl surfactant monomer d) may be present in an amount ranging from 0.01% to 25% by weight, such as from 0.1% to 10% by weight, relative to the total weight of the monomer mixture.

The at least one cationic polymer (i) used in the composition according to the present disclosure is prepared from a monomer mixture that may comprise at least one hydroxylated nonionic vinyl monomer e), which are, for example, ethylenically unsaturated monomers comprising at least one hydroxyl substituent.

Hydroxylated nonionic vinyl monomers e) that may be mentioned include, but are not limited to, hydroxylated C₁-C₆ alkyl (meth)acrylates, such as hydroxylated C₁-C₄ alkyl (meth)acrylates, 2-hydroxyethyl methacrylate (HEMA), 2-hydroxyethyl acrylate (2-HEA), and 3-hydroxypropyl acrylate; hydroxylated C₁-C₄ alkyl(meth)acrylamides, such as N-(2-hydroxyethyl)methacrylamide, N-(2-hydroxyethyl)acrylamide, N-(3-hydroxypropyl)acrylamide, and N-(2,3-dihydroxypropyl)acrylamide; and mixtures thereof. Mention may also be made, by way of non-limiting example, of allyl alcohol, glyceryl monoallyl ether, 3-methyl-3-buten-1-ol, and vinyl alcohol precursors, and equivalents thereof, such as vinyl acetate.

The at least one hydroxylated nonionic vinyl monomer e) may be present in an amount ranging from 0% to 10% by weight, relative to the total weight of the monomer mixture. In one embodiment, the at least one hydroxylated nonionic vinyl monomer e) is present in an amount ranging from 0.01% to 10% by weight, for example from 1% to 8% by weight, and further for example from 1% to 5% by weight, relative to the total weight of the monomer mixture.

The at least one cationic polymer (i) used in the composition according to the present disclosure is prepared from a monomer mixture that may comprise at least one crosslinking monomer for introducing branches and controlling the molecular mass.

Non-limiting examples of polyunsaturated crosslinking agents that may be used herein include monounsaturated compounds with a reactive group capable of crosslinking a copolymer formed before, during or after the polymerization may also be used; and polyfunctional monomers comprising multiple reactive groups such as peroxide and isocyanate groups and hydrolysable silane groups. Many polyunsaturated compounds may be used to generate a partially or substantially crosslinked three-dimensional network.

Examples of polyunsaturated crosslinking monomers that may be used include, but are not limited to, polyunsaturated aromatic monomers, such as divinylbenzene, divinyinaphthalene, and trivinylbenzene; polyunsaturated alicyclic monomers, such as 1,2,4-trivinylcyclohexane; difunctional phthalic acid esters, such as diallyl phthalate; polyunsaturated aliphatic monomers, such as dienes, trienes, and tetraenes, such as isoprene, butadiene, 1,5-hexadiene, 1,5,9-decatriene, 1,9-decadiene, and 1,5 heptadiene.

Other non-limiting examples of polyunsaturated crosslinking monomers that may be used, include polyalkenyl ethers such as triallylpentaerythritol, diallylpentaerythritol, diallylsucrose, octaallylsucrose, and trimethylolpropane diallyl ether; polyunsaturated esters of polyalcohols or of polyacids, such as 1,6-hexanediol di(meth)acrylate, tetramethylene tri(meth)acrylate, allyl acrylate, diallyl itaconate, diallyl fumarate, diallyl maleate, trimethylolpropane tri(meth)acrylate, trimethylolpropane di(meth)acrylate, and polyethylene glycol di(meth)acrylate; alkylenebisacrylamides, such as methylenebisacrylamide and propylenebisacrylamide; hydroxylated and carboxylated derivatives of methylenebisacrylamide, such as N,N′-bismethylol methylenebisacrylamide; polyethylene glycol di(meth)acrylates, such as ethylene glycol di(meth)acrylate, diethylene glycol di(meth)acrylate, and triethylene glycol di(meth)acrylate; polyunsaturated silanes, such as dimethyldivinylsilane, methyltrivinylsilane, allyidimethylvinylsilane, diallyldimethylsilane, and tetravinylsilane; and polyunsaturated stannanes, such as tetraallyltin and diallyldimethyltin.

Non-limiting examples of monounsaturated crosslinking monomers that may be used according to the present disclosure and that bear a reactive group include N-methylolacrylamides; N-alkoxy(meth)acrylamides, wherein the alkoxy group is a C₁-C₁₈ group; and unsaturated hydrolysable silanes such as triethoxyvinylsilane, tris-isopropoxyvinylsilane, and 3-triethoxysilylpropyl methacrylate.

Polyfunctional crosslinking monomers that may be used and that comprise several reactive groups may be, for example, hydrolysable silanes such as ethyltriethoxysilane and ethyltrimethoxysilane; epoxidized hydrolysable silanes such as 2-(3,4-epoxycyclohexyl)ethyltriethoxysilane and 3-glycidoxypropyltrimethyoxysilane; polyisocyanates, such as 1,4-diisocyanatobutane, 1,6-diisocyanatohexane, 1,4phenylenediisocyanate, and 4,4′-oxybis(phenyl)socyanate); unsaturated epoxides, such as glycidyl methacrylate and allyl glycidyl ether; polyepoxides, such as diglycidyl ether, 1,2,5,6-diepoxyhexane, and ethylene glycol diglycidyl ether.

In at least one embodiment, olyunsaturated crosslinking monomers that may be used herein include ethoxylated polyols, such as diols, triols, and bis-phenols, ethoxylated with 2 to 100 mol of ethylene oxide per mole of hydroxyl functional group and ending with a polymerizable unsaturated group such as a vinyl ether, an allyl ether, an acrylate ester, or a methacrylate ester. Such crosslinking monomers may be, for example, ethoxylated bisphenol A dimethacrylate, ethoxylated bisphenol F dimethacrylate, and ethoxylated trimethylolpropane trimethacrylate.

Other non-limiting examples of ethoxylated crosslinking monomers that may be used in the present disclosure include the crosslinking agents derived from ethoxylated polyols described in U.S. Pat. No. 6,140,435.

In at least one embodiment, examples of crosslinking monomers include, but are not limited to, acrylate and methacrylate esters of polyols comprising at least two acrylate or methacrylate ester groups, such as trimethylolpropane triacrylate (TMPTA), trimethylolpropane dimethacrylate, triethylene glycol dimethacrylate (TEGDMA), and ethoxylated (30) bisphenol A dimethacrylate (EOBDMA).

The at least one crosslinking monomer may be present in an amount ranging from 0% to 5% by weight, relative to the weight of the monomer mixture. According to one embodiment, the at least one crosslinking monomer is present in an amount ranging from 0.001% to 5% by weight, for example from 0.05% to 2% by weight, and further for example from 0.1% to 1% by weight, relative to the total weight of the monomer mixture.

The monomer mixture may comprise at least one chain-transfer agent. Non-limiting examples of the at least one chain-transfer agent include, but are not limited to, thiol compounds, disulfide compounds, such as C₁-C₁₈ mercaptans, mercaptocarboxylic acids, mercaptocarboxylic acid esters, thioesters, C₁-C₁₈ alkyl disulfides, aryl disulfides, polyfunctional thiols; phosphites and hypophosphites; haloalkyl compounds, such as carbon tetrachloride and bromotrichloromethane; and unsaturated chain-transfer agents, such as α-methylstyrene.

The polyfunctional thiols are, for example, trifunctional thiols, such as trimethylolpropane tris(3-mercaptopropionate), tetrafunctional thiols, such as pentaerythritol tetrakis(thioglycolate) and pentaerythritol tetrakis(thiolactate); hexafunctional thiols, such as pentaerythritol hexakis(thioglycolate).

Other non-limiting examples of the at least one chain-transfer agent include catalytic chain-transfer agents that reduce the molecular weight of the addition polymers during the free-radical polymerization of the vinyl monomers such as cobalt complexes, for example, cobalt (II) chelates. The at least one catalytic chain-transfer agent may also be used at low concentrations relative to the thiolated chain-transfer agents.

Non-limiting examples of the at least one chain-transfer agent that may be mentioned include octyl mercaptan, n-dodecyl mercaptan, t-dodecyl mercaptan, hexadecyl mercaptan, octadecyl mercaptan (ODM), isooctyl 3-mercaptopropionate (IMP), butyl 3-mercaptopropionate, 3-mercaptopropionic acid, butyl thioglycolate, isooctyl thioglycolate, and dodecyl thioglycolate.

The at least one chain-transfer agent may be present in an amount ranging from 0% to 10% by weight, relative to the total weight of the monomer mixture. In at least one embodiment, the at least one chain-transfer agent is present in an amount ranging from 0.1% to 5% by weight, relative to the total weight of monomers.

The monomer mixture for preparing the at least one cationic polymer (i) used in the composition according to the present disclosure may comprise at least one polymeric stabilizer for obtaining stable dispersions or emulsions. In one embodiment, the at least one polymer is water-soluble. Non-limiting examples include synthetic polymers, such as polyvinyl alcohols, partially hydrolysed polyvinyl acetates, polyvinylpyrrolidone, polyacrylamides, polymethacrylamides, carboxylated addition polymers, and polyalkyl vinyl ethers; water-soluble natural polymers, such as gelatin, pectins, alginates, and casein; and modified natural polymers, such as methylcellulose, hydroxypropylcellulose, carboxymethylcellulose, and allylic hydroxyethylcelluloses.

The at least one polymeric stabilizer may be present in an amount ranging from 0% to 2% by weight, relative to the total weight of the emulsion, for example in an amount of between 0.0001% and 1% by weight, and further for example between 0.01% and 0.5% by weight, relative to the weight of the monomer mixture.

According to one embodiment, the monomer mixture comprises, relative to the total weight of the monomer mixture:

a) from 10% to 70% by weight of at least one vinyl monomer substituted with at least one amino group a),

b) from 20% to 80% by weight of at least one hydrophobic nonionic vinyl monomer b),

c) from 0.001% to 25% by weight of at least one associative vinyl monomer c),

d) from 0 to 25% by weight of at least one semi-hydrophobic vinyl surfactant monomer d),

e) from 0 to 10% by weight of at least one hydroxylated nonionic vinyl monomer e),

f) from 0 to 5% by weight of at least one crosslinking monomer,

g) from 0 to 10% by weight of at least one chain-transfer agent, and

h) from 0 to 2% by weight of at least one polymeric stabilizer.

In another embodiment, the monomer mixture comprises, relative to the total weight of the monomer mixture:

a) from 20% to 60% by weight of at least one vinyl monomer substituted with at least one amino group a),

b) from 20% to 70% by weight of at least one hydrophobic nonionic vinyl monomer b),

c) from 0.01% to 15% by weight of at least one associative vinyl monomer c),

d) from 0.1% to 10% by weight of at least one semi-hydrophobic vinyl surfactant monomer d),

e) from 0.01% to 10% by weight of at least one hydroxylated nonionic vinyl monomer e),

f) from 0.001% to 5% by weight of at least one crosslinking monomer,

g) from 0.001% to 10% by weight of at least one chain-transfer agent, and

h) from 0 to 2% by weight of at least one polymeric stabilizer.

According to another embodiment, the monomer mixture for preparing the at least one cationic polymer (i) used in the composition according to the present disclosure comprises, relative to the total weight of the monomer mixture:

a) from 20% to 50% by weight of at least one vinyl monomer substituted with at least one amino group a) chosen from:
3-(N,N-dimethylamino)propyl (meth)acrylate,
N′-(3-N,N-dimethylamino)propyl(meth)acrylamide,
2-(N,N-dimethylamino)ethyl (meth)acrylate,
2-(N,N-diethylamino)ethyl (meth)acrylate,
2-(tert-butylamino)ethyl (meth)acrylate,
2-(N,N-dimethylamino)propyl(meth)acrylamide, and
2-(N,N-dimethylamino)neopentyl acrylate,

b) from 50% to 65% by weight of at least one hydrophobic nonionic vinyl monomer b) chosen from C₁-C₃₀ alkyl esters of acrylic acid, C₁-C₃₀ alkyl esters of methacrylic acid, and mixtures thereof,

c) from 0.1% to 10% by weight of at least one associative vinyl monomer c) chosen from polyethoxylated cetyl methacrylates, polyethoxylated cetearyl methacrylates, polyethoxylated stearyl (meth)acrylates, polyethoxylated arachidyl (meth)acrylates, polyethoxylated behenyl (meth)acrylates, polyethoxylated lauryl (meth)acrylates, polyethoxylated cerotyl (meth)acrylates, polyethoxylated montanyl (meth)acrylates, polyethoxylated melissyl (meth)acrylates, polyethoxylated lacceryl (meth)acrylates, polyethoxylated 2,4,6-tris(1′-phenylethyl)phenyl (meth)acrylates, polyethoxylated hydrogenated castor oil (meth)acrylates, polyethoxylated canola (meth)acrylates, and polyethoxylated cholesteryl (meth)acrylates, and mixtures thereof,

d) from 0.1% to 10% by weight of at least one semi-hydrophobic vinyl surfactant monomer d) chosen from one of the following formulae:

CH₂═CH—O(CH₂)ₐO(C₃H₆O)_{b}(C₂H₄O)_{c}H, and

CH₂═CHCH₂O(C₃H₆O)_{d}(C₂H₄O)ₑH;

wherein:

a is an integer ranging from 2 to 4;

b is an integer ranging from 1 to 10;

c is an integer ranging from 5 to 50;

d is an integer ranging from 1 to 10; and

e is an integer ranging from 5 to 50;

e) from 0% to 10% by weight of at least one hydroxylated nonionic vinyl monomer e),

f) from 0% to 5% by weight of at least one crosslinking monomer,

g) from 0% to 10% by weight of at least one chain-transfer agent, and

h) from 0% to 2% by weight of at least one polymeric stabilizer.

In one embodiment of the present disclosure, the at least one cationic polymer (i) is chosen from polymers derived from the polymerization of the following monomer mixture:

a di(C₁-C₄ alkyl)amino(C₁-C₆ alkyl)methacrylate,

at least one C₁-C₃₀ alkyl ester of (meth)acrylic acid,

a C₁₀-C₃₀ alkyl methacrylate polyethoxylated comprising from 20 to 30 mol of ethylene oxide,

a 30/5 polyethylene glycol/polypropylene glycol allyl ether,

a hydroxy(C₂-C₆ alkyl)methacrylate, and

an ethylene glycol dimethacrylate.

A non-limiting example of the at least one cationic polymer (i) used in the compositions disclosed herein includes the compound sold by the company Noveon under the name CARBOPOL Aqua CC Polymer and which corresponds to the INCl name Polyacrylate-1 Crosspolymer.

Polyacrylate-1 Crosspolymer is the product of polymerization of a monomer mixture comprising:

a di(C₁-C₄ alkyl)amino(C₁-C₆ alkyl)methacrylate,

at least one C₁-C₃₀ alkyl ester of (meth)acrylic acid,

a polyethoxylated C₁₀-C₃₀ alkyl methacrylate (20-25 mol of ethylene oxide units),

a 30/5 polyethylene glycol/polypropylene glycol allyl ether,

a hydroxy(C₂-C₆ alkyl)methacrylate, and

an ethylene glycol dimethacrylate.

The at least one cationic polymer (i) used in the compositions disclosed herein may be present in an amount ranging from 0.01% to 10% by weight, for example from 0.05% to 5% by weight, and further for example from 0.1% to 1% by weight, relative to the total weight of the composition.

The at least one cationic polymer (i) used in the composition according to the present disclosure may be prepared via conventional polymerization techniques, such as emulsion polymerization. The polymerization may be performed via a simple batch process, or via a controlled addition process, or the reaction may be initiated in a small reactor and the mass of monomers may then be added in a controlled manner to the reactor (seeding process). For example, the polymerization may be performed at a reaction temperature ranging from 20 to 80° C., although higher or lower temperatures may be used. To facilitate the emulsification of the monomer mixture, the emulsion polymerization is performed in the presence of a surfactant that is present in an amount ranging from 1% to 10% by weight, such as from 3% to 8% by weight, and further such as from 5% to 7% by weight, relative to the total weight of the emulsion. The emulsion polymerization reaction medium also comprises at least one radical initiator, which may be present, for example, in an amount ranging from 0.01% to 3% by weight, relative to the total weight of the monomer mixture. The polymerization may be performed in an aqueous or aqueous-alcoholic medium at a neutral or weakly alkaline pH.

In a typical polymerization, the monomer mixture is added with stirring to a solution of emulsifying surfactants, such as a nonionic surfactant, for instance a linear or branched alcohol ethoxylate, or a mixture of nonionic and anionic surfactants, such as fatty alkyl sulfates or alkyl sulfonates of fatty alcohols, in a suitable amount of water, in a suitable reactor, to prepare the monomer emulsion. The emulsion is deoxygenated via any known method, and the polymerization reaction is then initiated by adding a polymerization catalyst (initiator) such as sodium persulfate, or any other suitable addition polymerization catalyst, as is well known in the field of polymers. The reaction is stirred until the polymerization is complete, for example, for a time ranging from 4 hours to 16 hours. The monomer emulsion may be heated to a temperature ranging from 20 to 80° C. before adding the initiator, if so desired. The amount of unreacted monomers may be removed by adding an additional amount of catalyst. The polymer emulsion obtained may be discharged from the reactor and packaged for storage or used. Optionally, the pH or other physical or chemical characteristics of the emulsion may be adjusted before discharging the emulsion from the reactor. The emulsion produced can have a total solids content in an amount ranging between 10% and 40% by weight. The total amount of polymers in the emulsion obtained can range in an amount between 15% and 35% by weight, and, for example, not more than 25% by weight.

Surfactants that are suitable for facilitating the emulsion polymerization include, but are not limited to, surfactants conventionally used in emulsion polymerizations, such as nonionic, anionic, amphoteric, cationic surfactants, and mixtures thereof. In at least one embodiment, nonionic, anionic surfactants, and mixtures thereof are used.

The polymerization may be performed in the presence of at least one free-radical initiator chosen from, for example, insoluble inorganic persulfate compounds, such as ammonium persulfate, potassium persulfate, or sodium persulfate; peroxides, such as hydrogen peroxide, benzoyl peroxide, acetyl peroxide, and lauryl peroxide; organic hydroperoxides, such as cumene hydroperoxide and t-butyl hydroperoxide; organic peracids, such as peracetic acid; and oil-soluble free-radical generators, such as 2,2′-azobisisobutyronitrile, and mixtures thereof. The peroxides and peracids may be optionally activated with reducing agents, such as sodium bisulfite or ascorbic acid, transition metals, or hydrazine. The at least one free-radical initiator, in at least one embodiment, includes water-soluble azo polymerization initiators such as 2,2′-azobis(tert-alkyl) compounds bearing a water-solubilizing substituent on the alkyl group. Non-limiting examples of azo polymerization catalysts include the VAZO® free-radical initiators sold by the company DuPont, such as VAZO® 44 (2,2′-azobis(2-4,5-dihydroimidazolyl)propane), VAZO®56 (2,2′-azobis(2-methylpropionamidine) dihydrochloride), and VAZO® 68 (4,4′-azobis(4-cyanovaleric acid)).

The at least one triglyceride disclosed herein is, for example, insoluble in water at a concentration of greater than or equal to 0.1% by weight in water at 25° C., i.e., they do not form under these conditions an isotropic solution that is transparent to the naked eye.

The at least one triglyceride according to the present disclosure is, for instance, chosen from triglycerides of sweet almond oil, avocado oil, castor oil, olive oil, sesameseed oil, groundnut oil, rapeseed oil, canola oil, macadamia nut oil, coconut oil, hazelnut oil, cashew nut oil, yellow flax (camelina) oil, shea butter, palm oil, apricot kernel oil, argan oil, beauty-leaf oil, and grapeseed oil, and mixtures thereof.

In one embodiment, the at least one triglyceride is chosen, for example, from the group formed by the triglycerides of avocado oil, olive oil, apricot kernel oil, coconut oil, palm oil, and argan oil, and mixtures thereof.

The at least one triglyceride according to the present disclosure may be present in an amount ranging from 0.01% to 20% by weight, relative to the total weight of the composition. In at least one embodiment, this amount ranges from 0.05% to 15% by weight, such as from 0.1% to 10% by weight, relative to the total weight of the composition.

According to the present disclosure, the composition comprises at least one non-silicone polymer (iii) other than the at least one cationic polymer (i), chosen, for example, from non-silicone cationic polymers, non-silicone amphoteric polymers, and mixtures thereof.

The at least one cationic polymer (iii) that may be used in accordance with the present disclosure may be chosen from all those already known to improve the cosmetic properties of the hair, for instance those described in European Patent Application No. 0 337 354 and in French Patent Application Nos. 2 270 846, 2 383 660, 2 598 611, 2 470 596, and 2 519 863.

As used herein, “cationic polymer” is understood to mean any polymer comprising cationic groups and/or groups that may be ionized into cationic groups.

In at least one embodiment, the at least one cationic polymer (iii) is chosen from those containing units comprising primary, secondary, tertiary, and/or quaternary amine groups that either may form part of the main polymer chain or may be borne by a side substituent directly attached thereto.

The at least one cationic polymer (iii) disclosed herein may have, for example, a number-average or weight-average molecular mass of between approximately 500 and 5×10⁶ such as between approximately 10³ and 3×10⁶.

Non-limiting examples of the at least one cationic polymer (iii) that may be mentioned include polymers of the polyamine, polyamino amide, and polyquaternary ammonium type, such as those described in French Patent Nos. 2 505 348 and 2 542 997. Among these polymers, non-limiting mention may be made of:

(1) homopolymers or copolymers derived from acrylic or methacrylic esters or amides and comprising at least one of the units of the following formulae:

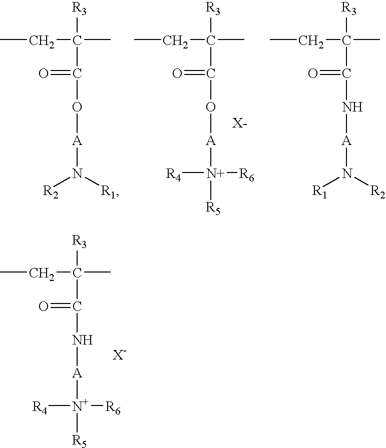

wherein:
R₃, which may be identical or different, is chosen from a hydrogen atom and a CH₃ radical; A, which may be identical or different, is chosen from a C₁-C₆ linear and branched alkyl group, such as a C₂-C₃ linear and branched alkyl group, and a C₁-C₄ hydroxyalkyl group;
R₄, R₅, and R₆, which may be identical or different, are chosen from C₁-C₁₈ alkyl groups and benzyl radicals, such as C₁-C₆ alkyl groups;
R₁ and R₂, which may be identical or different, are chosen from hydrogen atoms and C₁-C₆ alkyl groups, such as methyl or ethyl;
X is an anion derived from a mineral or organic acid, such as a methosulfate anion or a halide such as chloride or bromide.

The copolymers of family (1) can also comprise at least one unit derived from comonomers chosen from, by way of non-limiting example, acrylamides, methacrylamides, diacetone acrylamides, acrylamides, and methacrylamides substituted on the nitrogen with lower (C₁-C₄) alkyls, acrylic or methacrylic acids or esters thereof, vinyllactams such as vinylpyrrolidone or vinylcaprolactam, and vinyl esters.

Thus, among these copolymers of family (1), non-limiting mention may be made of:

copolymers of acrylamide and of dimethylaminoethyl methacrylate quaternized with dimethyl sulfate or with a dimethyl halide, such as the product sold under the name HERCOFLOC by the company Hercules,

the copolymers of acrylamide and of methacryloyloxyethyltrimethylammonium chloride described, for example, in European Patent Application No. 0 080 976 and sold under the name BINA QUAT P 100 by the company Ciba Geigy,

the copolymer of acrylamide and of methacryloyloxyethyltrimethylammonium methosulfate sold, for instance, under the name RETEN by the company Hercules,

quaternized or nonquaternized vinylpyrrolidone/dialkylaminoalkyl acrylate or methacrylate copolymers, such as the products sold, for example, under the name GAFQUAT by the company ISP, such as GAFQUAT® 734 or GAFQUAT® 755, or alternatively the products known as COPYLMER 845, 958, and 937. These polymers are described, for example, in French Patent Nos. 2 077 143 and 2 393 573,

dimethylaminoethyl methacrylate/vinylcaprolactam/vinylpyrrolidone terpolymers, such as the product sold, for example, under the name GAFFIX® VC 713 by the company ISP,

vinylpyrrolidone/methacrylamidopropyldimethylamine copolymers sold, for instance, under the name STYLEZE® CC 10 by ISP,

quaternized vinylpyrrolidone/dimethylaminopropylmethacrylamide copolymers, such as the product sold, for example, under the name GAFQUAT® HS 100 by the company ISP, and

crosslinked methacryloyloxy(C₁-C₄)alkyltri(C₁-C₄)alkylammonium salt polymers such as the polymers obtained by homopolymerization of dimethylaminoethyl methacrylate quaternized with methyl chloride, or by copolymerization of acrylamide with dimethylaminoethyl methacrylate quaternized with methyl chloride, the homo- or copolymerization being followed by crosslinking with a compound containing olefinic unsaturation, such as methylenebisacrylamide. A crosslinked acrylamide/methacryloyloxyethyltrimethylammonium chloride copolymer (20/80 by weight) in the form of a dispersion comprising 50% by weight of the said copolymer in mineral oil can be used, for instance, the dispersion sold under the name SALCARE® SC 92 by the company Ciba. A crosslinked methacryloyloxyethyltrimethylammonium chloride homopolymer comprising about 50% by weight of the homopolymer in mineral oil or in a liquid ester can also be used. These dispersions are sold, for instance, under the names SALCARE® SC 95 and SALCARE® SC 96 by the company Ciba.

(2) cationic polysaccharides, such as cationic celluloses and cationic galactomannan gums. Other non-limiting examples of cationic polysaccharides that may be mentioned include cellulose ether derivatives comprising quaternary ammonium groups, cationic cellulose copolymers, or cellulose derivatives grafted with a water-soluble quaternary ammonium monomer and cationic galactomannan gums.

The cellulose ether derivatives comprising quaternary ammonium groups, which are described, for instance, in French Patent No. 1 492 597, such as the polymers sold under the names JR (JR 400, JR 125, JR 30M) or LR (LR 400, LR 30M) by the company Nalco. These polymers are also defined in the CTFA dictionary as hydroxyethylcellulose quaternary ammoniums that have reacted with an epoxide substituted with a trimethylammonium group.

The cationic cellulose copolymers or cellulose derivatives grafted with a water-soluble quaternary ammonium monomer are described, for example, in U.S. Pat. No. 4,131,576, such as hydroxyalkylcelluloses, for instance hydroxymethyl-, hydroxyethyl-, or hydroxypropylcelluloses grafted, for example, with a methacryloyl-ethyltrimethylammonium, methacrylamidopropyltrimethylammonium, or dimethyldiallylammonium salt.

The commercial products corresponding to this definition include, but are not limited to, the products sold under the names CELQUAT® L 200 and CELQUAT® H 100 by the company National Starch.

The cationic galactomannan gums are described, for example, in U.S. Pat. Nos. 3,589,578 and 4,031,307, such as guar gums comprising trialkylammonium cationic groups. Use is made, for example, of guar gums modified with a salt (e.g. chloride) of 2,3-epoxypropyltrimethylammonium.

Such products are sold, for instance under the trade names JAGUAR® C13S, JAGUAR® C 15, JAGUAR® C 17, or JAGUAR® C162 by the company Rhodia Chimie.

(3) polymers comprising piperazinyl units and divalent alkylene or hydroxyalkylene radicals comprising straight or branched chains, optionally interrupted by oxygen, sulfur, or nitrogen atoms or by aromatic or heterocyclic rings, as well as the oxidation and/or quaternization products of these polymers, such as the polymers described, for example, in French Patent Nos. 2 162 025 and 2 280 361;

(4) water-soluble polyamino amides prepared, for example, by polycondensation of an acidic compound with a polyamine; these polyamino amides can be crosslinked with an epihalohydrin, a diepoxide, a dianhydride, an unsaturated dianhydride, a bis-unsaturated derivative, a bis-halohydrin, a bis-azetidinium, a bis-haloacyldiamine, a bis-alkyl halide, or with an oligomer resulting from the reaction of a difunctional compound which is reactive with a bis-halohydrin, a bis-azetidinium, a bis-haloacyldiamine, a bis-alkyl halide, an epihalohydrin, a diepoxide, or a bis-unsaturated derivative; the crosslinking agent being used in an amount ranging from 0.025 to 0.35 mol per amine group of the polyamino amide; these polyamino amides can be alkylated or, if they comprise at least one tertiary amine function, they can be quaternized. Such polymers are described, for instance, in French Patent Nos. 2 252 840 and 2 368 508;

(5) polyaminoamide derivatives resulting from the condensation of polyalkylene polyamines with polycarboxylic acids followed by alkylation with difunctional agents, such as adipic acid/dialkylaminohydroxyalkyldialkylenetriamine polymers wherein the alkyl radical comprises from 1 to 4 carbon atoms, such as methyl, ethyl, and propyl, and the polymers described, for instance, in French Patent No. 1 583 363.

Among these derivatives, mention may be, in a non-limiting manner, of the adipic acid/dimethylaminohydroxypropyl/diethylenetriamine polymers sold under the name CARTARETINE F, F4, or F8 by the company Sandoz.

(6) polymers obtained by reaction of a polyalkylene polyamine comprising two primary amine groups and at least one secondary amine group with a dicarboxylic acid chosen from diglycolic acid and saturated aliphatic dicarboxylic acids having from 3 to 8 carbon atoms, wherein the molar ratio between the polyalkylene polyamine and the dicarboxylic acid being between 0.8:1 and 1.4:1; the polyamino amide resulting therefrom being reacted with epichlorohydrin in a molar ratio of epichlorohydrin relative to the secondary amine group of the polyamino amide of between 0.5:1 and 1.8:1. Such polymers are described, for example, in U.S. Pat. Nos. 3,227,615 and 2 961 347.

Polymers of this type are sold, for example, under the name HERCOSETT 57 by the company Hercules Inc. or under the names PD 170 or DELSETTE 101 by the company Hercules in the case of the adipic acid/epoxypropyl/diethylenetriamine copolymer.

(7) cyclopolymers of alkyldiallylamine or of dialkyldiallylammonium, such as the homopolymers or copolymers comprising, as main constituent of the chain, units of formula (VI) or (VII):

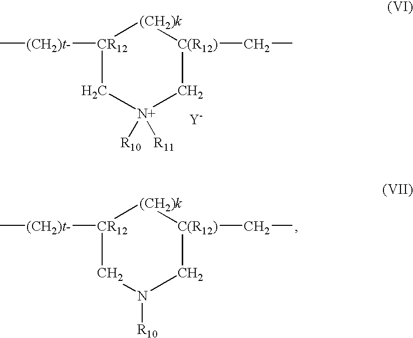

wherein formulae k and t are integers ranging from 0 to 1, the sum k+t being 1; R₁₂ is chosen from a hydrogen atom and a methyl radical; R₁₀ and R₁₁ are independently chosen from C₁-C₆ alkyl groups, C₁-C₅ hydroxyalkyl groups, lower C₁-C₄ amidoalkyl groups, or R₁₀ and R₁₁ can form, together with the nitrogen atom to which they are attached, heterocyclic groups such as piperidyl or morpholinyl; Y is an anion such as bromide, chloride, acetate, borate, citrate, tartrate, bisulfate, bisulfite, sulfate, and phosphate. These polymers are described, for example, in French Patent No. 2 080 759 and in its Certificate of Addition No. 2 190 406.

R₁₀ and R₁₁, independently of each other, are chosen from C₁-C₄ alkyl groups.

Among the polymers defined above, non-limiting mention may be made, for example, of the dimethyldiallylammonium chloride homopolymer sold, for instance, under the name MERQUAT® 100 by the company Nalco (and its homologues of low weight-average molecular mass) and copolymers of diallyidimethylammonium chloride and of acrylamide, sold for example under the name MERQUAT® 550.

(8) quaternary diammonium polymers comprising repeating units corresponding to the formula:

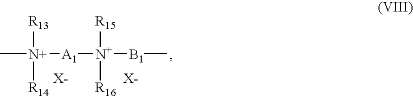

wherein:

R₁₃, R₁₄, R₁₅, and R₁₆, which may be identical or different, are chosen from aliphatic, alicyclic, and arylaliphatic radicals comprising from 1 to 20 carbon atoms and lower hydroxyalkylaliphatic radicals, or R₁₃, R₁₄, R₁₅, and R₁₆, together or separately, constitute, with the nitrogen atoms to which they are attached, heterocycles optionally comprising a second hetero atom other than nitrogen, or R₁₃, R₁₄, R₁₅, and R₁₆ are chosen from linear and branched C₁-C₆ alkyl radicals substituted with a nitrile, ester, acyl, or amide group or a —CO—O—R₁₇-D or —CO—NH—R₁₇-D group wherein R₁₇ is an alkylene and D is a quaternary ammonium group;

A₁ and B₁ are chosen from polymethylene groups comprising from 2 to 20 carbon atoms, which may be linear or branched, saturated or unsaturated, and which may comprise, linked to or intercalated in the main chain, at least one aromatic ring or at least one oxygen or sulfur atom, or sulfoxide, sulfone, disulfide, amino, alkylamino, hydroxyl, quaternary ammonium, ureido, amide, or ester group, and

X⁻ is an anion derived from an inorganic or organic acid;

A₁, R₁₃, and R₁₅ can form, with the two nitrogen atoms to which they are attached, a piperazine ring; in addition, if A₁ is a linear or branched, saturated or unsaturated alkylene or hydroxyalkylene radical, B₁ can also be (CH₂)ₙ—CO-D-OC—(CH₂)ₚ—;

wherein:

n and p, which may be identical or different, are integers ranging from 2 to 20,

D is chosen from:

a) a glycol residue of formula: —O-Z-O—, wherein Z is chosen from a linear and branched hydrocarbon-based radical and a group of one of the following formulae:

—(CH₂—CH₂—O)ₓ—CH₂—CH₂—; and

—[CH₂—CH(CH₃)—O]_{y}—CH₂—CH(CH₃)—,

wherein x and y are integers ranging from 1 to 4, representing a defined and unique degree of polymerization or any integer ranging from 1 to 4 representing an average degree of polymerization;

b) a bis-secondary diamine residue such as a piperazine derivative;

c) a bis-primary diamine residue of formula: —NH—Y—NH—, wherein Y is chosen from a linear and branched hydrocarbon-based radical, and a divalent radical of formula:

—CH₂—CH₂—S—S—CH₂—CH₂—; and

d) a ureylene group of formula: —NH—CO—NH—;

in at least one embodiment, X⁻ is an anion such as chloride or bromide.

As disclosed herein, these polymers have, for example, a number-average molecular mass of between 1000 and 100 000.

Polymers of this type are described, for example, in French Patent Nos. 2 320 330, 2 270 846, 2 316 271, 2 336 434 and 2 413 907 and U.S. Pat. Nos. 2,273,780, 2,375,853, 2,388,614, 2,454,547, 3,206,462, 2,261,002, 2,271,378, 3,874,870, 4,001,432, 3,929,990, 3,966,904, 4,005,193, 4,025,617, 4,025,627, 4,025,653, 4,026,945, and 4,027,020.

Other polymers that may be used herein comprise repeating units of formula (a):

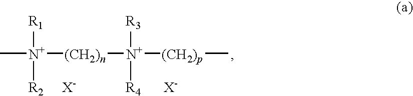

wherein R₁, R₂, R₃, and R₄, which may be identical or different, are chosen from alkyl and hydroxyalkyl radicals comprising 1 to 4 carbon atoms, n and p are integers ranging from 2 to 20, and X⁻ is an anion derived from an inorganic or organic acid.

In at least one embodiment, R₁, R₂, R₃, and R₄ are methyl radicals and n is 3, p is 6, and X is Cl, which is known as Hexadimethrine chloride according to the INCl (CTFA) nomenclature.

(9) polyquaternary ammonium polymers comprising units of formula (IX):

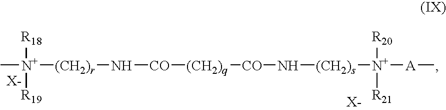

wherein:

R₁₈, R₁₉, R₂₀ and R₂₁ which may be identical or different, are chosen from hydrogen atoms, and methyl, ethyl, propyl, β-hydroxyethyl, β-hydroxypropyl, and

—CH₂CH₂(OCH₂CH₂)ₚOH radicals,

wherein p is an integer ranging from 0 to 6, with the proviso that R₁₈, R₁₉, R₂₀, and R₂₁ are not simultaneously hydrogen atoms,

r and s, which may be identical or different, are integers ranging from 1 to 6,

q is an integer ranging from 0 to 34,

X⁻ is an anion such as a halide,

A is chosen from a dihalide radical and —CH₂—CH₂—O—CH₂—CH₂—.

Such compounds are described, for example, in European Patent Application No. 0 122 324.

Among these products, non-limiting mention may be made, for example, of the products MIRAPOL®A 15, MIRAPOL® AD1, MIRAPOL®DAZ1, and MIRAPOL® 175 sold by the company Miranol.

(10) quaternary polymers of vinylpyrrolidone and of vinylimidazole, for instance the products sold under the names LUVIQUAT® FC 905, FC 550, and FC 370 by the company BASF.

(11) polyamines, for instance POLYQUART® H sold by Cognis, referenced under the name Polyethylene Glycol (15) Tallow Polyamine in the CTFA dictionary.

Other cationic polymers (iii) that can be used in the context of the present disclosure include, but are not limited to, cationic proteins, cationic protein hydrolysates, polyalkyleneimines, such as polyethyleneimines, polymers comprising vinylpyridine or vinylpyridinium units, condensates of polyamines and of epichlorohydrin, quaternary polyureylenes, and chitin derivatives.

According to at least one embodiment of the present disclosure, the at least one cationic polymer (iii) may be a quaternary cellulose ether derivative such as the products sold, for example, under the name JR 400 by the company Nalco, cationic cyclopolymers, such as the dimethyldiallylammonium chloride homopolymers or copolymers sold under the names MERQUAT 100, MERQUAT 550 and MERQUAT S by the company Nalco, quaternary polymers of vinylpyrrolidone and of vinylimidazole, crosslinked homopolymers or copolymers of methacryloyloxy(C₁-C₄)alkyltri(C₁-C₄)alkylammonium salts, and mixtures thereof.

The amphoteric polymers that may be used in accordance with the present disclosure may be chosen from polymers comprising units K and M randomly distributed in the polymer chain, wherein K is a unit derived from a monomer comprising at least one basic nitrogen atom and M is a unit derived from an acidic monomer comprising at least one carboxylic or sulfonic group, or K and M may be groups derived from zwitterionic carboxybetaine or sulfobetaine monomers;

K and M may also be a cationic polymer chain comprising primary, secondary, tertiary, or quaternary amine groups, wherein at least one of the amine groups bears a carboxylic or sulfonic group linked via a hydrocarbon-based radical, or K and M form part of a chain of a polymer comprising an α,β-dicarboxylic ethylene unit wherein one of the carboxylic groups has been made to react with a polyamine comprising at least one primary or secondary amine group.

Examples of amphoteric suitable polymers corresponding to the above definition include, but are not limited to:

(1) polymers resulting from the copolymerization of a monomer derived from a vinyl compound bearing a carboxylic group such as acrylic acid, methacrylic acid, maleic acid, α-chloroacrylic acid, and a basic monomer derived from a substituted vinyl compound comprising at least one basic atom, such as dialkylaminoalkyl methacrylate and acrylate, dialkylaminoalkylmethacrylamide, and -acrylamide. Such compounds are described, for example, in U.S. Pat. No. 3,836,537. Non-limiting mention may also be made of the sodium acrylate/acrylamidopropyltrimethylammonium chloride copolymer sold, for instance, under the name POLYQUART KE 3033 by the company Cognis.

The vinyl compound may also be a dialkyldiallylammonium salt such as dimethyldiallylammonium chloride. The copolymers of acrylic acid and of the latter monomer are sold, for instance, under the names MERQUAT 280, MERQUAT 295, and MERQUAT Plus 3330 by the company Nalco.

(2) Polymers comprising units derived from:

a) at least one monomer chosen from acrylamides and methacrylamides substituted on the nitrogen with an alkyl radical,

b) at least one acidic comonomer comprising at least one reactive carboxylic group, and

c) at least one basic comonomer such as esters comprising primary, secondary, tertiary, and quaternary amine substituents of acrylic and methacrylic acids and the product of quaternization of dimethylaminoethyl methacrylate with dimethyl or diethyl sulfate.

Useful examples of N-substituted acrylamides or methacrylamides include, but are not limited to, C₂-C₁₂ alkyl radicals, such as N-ethylacrylamide, N-tert-butyl-acrylamide, N-tert-octylacrylamide, N-octylacrylamide, N-decylacrylamide, N-dodecylacrylamide, and methacrylamides.

The acidic comonomers are chosen, for example, from acrylic acid, methacrylic acid, crotonic acid, itaconic acid, maleic acid and fumaric acid, and alkyl monoesters, comprising 1 to 4 carbon atoms, of maleic or fumaric acids or anhydrides.

The basic comonomers are, for instance, aminoethyl, butylaminoethyl, N,N′-dimethylaminoethyl, and N-tert-butylaminoethyl methacrylates.

The copolymers whose CTFA (4th edition, 1991) name is Octylacrylamide/acrylates/butylaminoethyl methacrylate copolymer such as the products sold, for example, under the name AMPHOMER or LOVOCRYL 47 by the company National Starch may be used.

(3) Polyamino amides that are crosslinked and alkylated partially or totally derived from polyamino amides of general formula:

CO—R₄—CO-Z (X)

wherein R₄ is chosen from divalent radicals derived from a saturated dicarboxylic acid, mono- and dicarboxylic aliphatic acids comprising an ethylenic double bond, an ester of a lower alkanol, having 1 to 6 carbon atoms of these acids and radicals derived from the addition of any one of said acids to a bis(primary) or bis(secondary) amine, and Z is chosen from bis(primary), mono- and bis(secondary) polyalkylene-polyamine radicals such as radicals:

a) in proportions of from 60 to 100 mol %, the radical

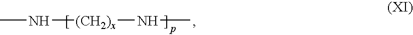

wherein x is 2 and p is 2 or 3, or x is 3 and p is 2
this radical being derived from diethylenetriamine, from triethylenetetraamine, or from dipropylenetriamine;

b) in proportions of from 0 to 40 mol %, the radical (V) above wherein x is 2 and p is 1 and which is derived from ethylenediamine, or the radical derived from piperazine:

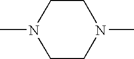

c) in proportions of from 0& to 20 mol %, the —NH—(CH₂)₆—NH— radical derived from hexamethylenediamine, these polyamino amines being crosslinked by addition of a difunctional crosslinking agent chosen from epihalohydrins, diepoxides, dianhydrides, and bis-unsaturated derivatives, using from 0.025 to 0.35 mol of crosslinking agent per amine group of the polyamino amide and alkylated by the action of acrylic acid, chloroacetic acid, or an alkane sultone, or salts thereof.

The saturated carboxylic acids are, in at least one embodiment, chosen from acids having 6 to 10 carbon atoms, such as adipic acid, 2,2,4-trimethyladipic acid and 2,4,4-trimethyladipic acid, terephthalic acid and acids comprising an ethylenic double bond such as, for example, acrylic acid, methacrylic acid, and itaconic acid.

The alkane sultones used in the alkylation are, for example, propane sultone or butane sultone, and the salts of the alkylating agents are, for instance, the sodium or potassium salts.

(4) Polymers containing zwitterionic units of formula:

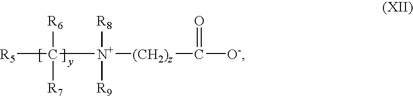

wherein R₅ is a polymerizable unsaturated group such as an acrylate, methacrylate, acrylamide, or methacrylamide group, y and z are integers ranging from 1 to 3, R₆ and R₇ are chosen from hydrogen atoms, methyl, ethyl, and propyl groups, R₈ and R₉ are chosen from hydrogen atoms and alkyl radicals such that the sum of the carbon atoms in R₈ and R₉ does not exceed 10.

The polymers comprising such units may also comprise units derived from non-zwitterionic monomers such as dimethyl and diethylaminoethyl acrylate and methacrylate, alkyl acrylates and methacrylates, acrylamides and methacrylamides, and vinyl acetate.

By way of example, non-limiting mention may be made of the copolymer of butyl methacrylateldimethyl carboxymethylammonio ethyl methacrylate.

(5) Polymers derived from chitosan comprising monomer units of formulae (XIII), (XIV), and (XV).

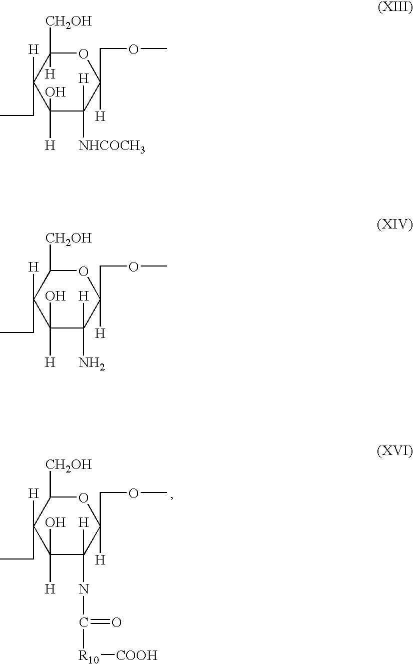

wherein the unit (XIII) may be present in an amount ranging from 0% and 30%, the unit (XIV) may be present in an amount ranging from 5% and 50%, and the unit (XV) may be present in an amount ranging from 30% and 90%, it being understood that, in this unit (XV), R₁₀ is a radical of formula:

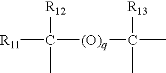

wherein
if q is 0, R₁₁, R₁₂, and R₁₃, which may be identical or different, are chosen from hydrogen atoms, methyl, hydroxyl, acetoxy, amino residues, monoalkylamine residues, and dialkylamine residues, which are optionally interrupted by at least one nitrogen atom and/or optionally substituted with at least one group chosen from amine, hydroxyl, carboxyl, alkylthio and sulfonic groups, and alkylthio residues, wherein the alkyl group bears an amino residue, wherein at least one of the radicals R₁₁, R₁₂, and R₁₃ being, in this case, a hydrogen atom;
or, if q is 1, R₁₁, R₁₂, and R₁₃ are each a hydrogen atom, and the salts formed by these compounds with bases or acids.

(6) Polymers derived from the N-carboxyalkylation of chitosan, such as N-carboxymethylchitosan or N-carboxybutylchitosan.

(7) Polymers corresponding to the general formula (XVI) such as those described, in French Patent No. 1 400 366:

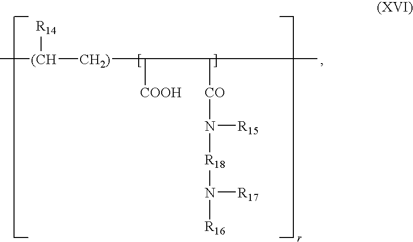

wherein R₁₄ is chosen from a hydrogen atom, CH₃O, CH₃CH₂O, and a phenyl radical, R₁₅ is chosen from a hydrogen atom and a lower alkyl radical, such as methyl or ethyl, R₁₆ is chosen from a hydrogen atom and a lower alkyl radical, such as methyl or ethyl, R₁₇ is chosen from a lower alkyl radical, such as methyl or ethyl or a radical corresponding to the formula: —R₁₈—N(R₁₆)₂, wherein R₁₈ is chosen from —CH₂—CH₂—, —CH₂—CH₂—CH₂—, and —CH₂—CH(CH₃)—, and R₁₆ has the meaning defined above, and also the higher homologues of these radicals and comprising up to 6 carbon atoms.

(8) Amphoteric polymers of the type -D-X-D-X— chosen from:

a) polymers obtained by the action of chloroacetic acid or sodium chloroacetate on compounds comprising at least one unit of formula:

-D-X-D-X-D- (XVII)

wherein D is a radical

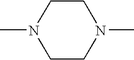

and X is the symbol E or E′, wherein E or E′, which may be identical or different, are chosen from divalent radicals, which is an alkylene radical with a straight or branched chain comprising up to 7 carbon atoms in the main chain, which is unsubstituted or substituted with hydroxyl groups and which can comprise, in addition to the oxygen, nitrogen, and sulfur atoms, 1 to 3 aromatic and/or heterocyclic rings; the oxygen, nitrogen, and sulfur atoms being present in the form of ether, thioether, sulfoxide, sulfone, sulfonium, alkylamine or alkenylamine groups, hydroxyl, benzylamine, amine oxide, quaternary ammonium, amide, imide, alcohol, ester, and/or urethane groups;

b) polymers of formula:

-D-X-D-X— (XVII)

wherein D is a radical

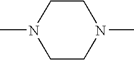

and X is the symbol E or E′ and in at least one embodiment E′; wherein E has the meaning given above and E′ is a divalent radical chosen from an alkylene radical with a straight or branched chain comprising up to 7 carbon atoms in the main chain, which is unsubstituted or substituted with at least one hydroxyl radical and comprising at least one nitrogen atom, the nitrogen atom being substituted with an alkyl chain which is optionally interrupted by an oxygen atom and necessarily comprising at least one carboxyl function or at least one hydroxyl function and betainized by reaction with chloroacetic acid or sodium chloroacetate.

(9) (C₁-C₅)alkyl vinyl ether/maleic anhydride copolymers partially modified by semiamidation with an N,N-dialkylaminoalkylamine such as N,N-dimethylaminopropylamine or by semiesterification with an N,N-dialkanolamine. These copolymers can also comprise other vinyl comonomers such as vinylcaprolactam.

The amphoteric polymers that may be used in at least one embodiment of to the present disclosure are those of family (1).

According to the present disclosure, the at least one cationic or amphoteric polymer may be present in an amount ranging from 0.001% to 20% by weight, for example from 0.01% to 10% by weight, and further for example from 0.1% to 5% by weight, relative to the total weight of the final composition.

The compositions disclosed herein may further comprise at least one surfactant, which is present in an amount ranging from approximately 0.01% to 50% by weight, for example from 0.1% to 40% by weight, and further for example from 0.5% to 30% by weight, relative to the total weight of the composition.

This surfactant may be chosen from anionic, amphoteric, nonionic, cationic surfactants, and mixtures thereof.

Suitable examples of the at least one surfactant include, but are not limited to:

(i) Anionic Surfactants:

Non-limiting examples of anionic surfactants which can be used, alone or as mixtures, include salts, for example, alkaline salts, such as sodium salts, ammonium salts, amine salts, amino alcohol salts, and magnesium salts of the following compounds: alkyl sulfates, alkyl ether sulfates, alkylamidoether sulfates, alkylarylpolyether sulfates, monoglyceride sulfates; alkyl sulfonates, alkyl phosphates, alkylamide sulfonates, alkylaryl sulfonates, α-olefin sulfonates, paraffin sulfonates; alkyl sulfosuccinates, alkyl ether sulfosuccinates, alkylamide sulfosuccinates; alkyl sulfosuccinamates; alkyl sulfoacetates; alkyl ether phosphates; acyl sarcosinates; acyl isethionates and N-acyltaurates, the alkyl or acyl radical of all of these various compounds comprise, in at least one embodiment, from 8 to 24 carbon atoms, and the aryl radical is chosen from a phenyl and benzyl group, for example, the fatty acid salts such as the salts of oleic, ricinoleic, palmitic and stearic acids, coconut oil acid, or hydrogenated coconut oil acid; acyl lactylates wherein the acyl radical comprises 8 to 20 carbon atoms. Weakly anionic surfactants can also be used, such as alkyl-D-galactosiduronic acids and their salts, as well as polyoxyalkylenated (C₆-C₂₄) alkyl ether carboxylic acids, polyoxyalkylenated (C₆-C₂₄) alkylaryl ether carboxylic acids, polyoxyalkylenated (C₅-C₂₄) alkylamido ether carboxylic acids, and their salts, such as those comprising from 2 to 50 ethylene oxide groups, and mixtures thereof.

Anionic surfactants that may be used according to the present disclosure include, but are not limited to, alkyl sulfate salts and alkyl ether sulfate salts, and mixtures thereof, such as sodium lauryl sulfate, ammonium lauryl sulfate, sodium lauryl ether sulfate, ammonium lauryl ether sulfate, and magnesium lauryl ether sulfate.

(ii) Nonionic Surfactants:

The nonionic surfactants are, for instance, compounds described in the “Handbook of Surfactants” by M. R. Porter, published by Blackie & Son (Glasgow and London), 1991, pp. 116-178 Thus, they can be chosen, for example from polyethoxylated, polypropoxylated and polyglycerolated fatty acids, alkylphenols, α-diols and alcohols having a fatty chain comprising, for example, 8 to 18 carbon atoms, it being possible for the number of ethylene oxide or propylene oxide groups to range, for example, from 2 to 50 and for the number of glycerol groups to range, for instance, from 2 to 30. Non-limiting mention may also be made of copolymers of ethylene oxide and of propylene oxide, condensates of ethylene oxide and of propylene oxide with fatty alcohols; polyethoxylated fatty amides comprising, for example, from 2 to 30 mol of ethylene oxide, polyglycerolated fatty amides comprising on average 1 to 5, such as 1.5 to 4, glycerol groups; polyethoxylated fatty amines comprising, for instance 2 to 30 mol of ethylene oxide; oxyethylenated fatty acid esters of sorbitan comprising from 2 to 30 mol of ethylene oxide; fatty acid esters of sucrose, fatty acid esters of polyethylene glycol, alkylpolyglycosides, N-alkylglucamine derivatives, amine oxides such as (C₁₀-C₁₄)alkylamine oxides or N-acylaminopropylmorpholine oxides. In at least one embodiment, the alkylpolyglycosides, such as (C₁₂-C₁₄)alkylpolyglucosides may be used.

(iii) Amphoteric Surfactants:

The amphoteric surfactants that may by used herein include, by way of non-limiting example, aliphatic secondary and tertiary amine derivatives, wherein the aliphatic radical is chosen from linear and branched chain comprising 8 to 22 carbon atoms and comprising at least one water-soluble anionic group, for example, carboxylate, sulfonate, sulfate, phosphate, and phosphonate; non-limiting mention may also be made of (C₈-C₂₀))alkylbetaines, sulfobetaines, (C₈-C₂₀)alkylamido(C₁-C₆)alkylbetaines, and (C₈-C₂₀)alkylamido(C₁-C₆)alkylsulfobetaines.

Among the (C₈-C₂₀)alkylamido(C₁-C₆)alkylbetaines that may be mentioned include, but are not limited to, cocoamidopropylbetaine sold, for instance by Goldschmidt under the name TEGOBETAINE F50.

Among the amine derivatives, mention may be made, by way of non-limiting example, of the products sold under the name MIRANOL, as described in U.S. Pat. Nos. 2,528,378 and 2,781,354 and having the structures:

R₂—CONHCH₂CH₂—N(R₃)(R₄)(CH₂COO—) (2)

wherein: R₂ is chosen from an alkyl radical derived from an acid R₂—COOH present in hydrolysed coconut oil, a heptyl, nonyl, or undecyl radical, R₃ is a β-hydroxyethyl group, and R₄ is a carboxymethyl group;

and

R₅—CONHCH₂CH₂—N(B)(C) (3)

wherein:

B is —CH₂CH₂OX′, C is —(CH₂)ₙ—Y′, with z=1 or 2,

X′ is chosen from —CH₂CH₂—COOH and a hydrogen atom,

Y′ is chosen from —COOH and a —CH₂—CHOH—SO₃H radical, and

R₅ is chosen from an alkyl radical of an acid R₉—COOH present in coconut oil or in hydrolysed linseed oil, and alkyl radicals such as a C₇, C₉, C₁₁ and C₁₃ alkyl radical, a C₁₋₇ alkyl radical and its iso form, and an unsaturated C₁₇ radical; and R₉ is chosen from alkyl radicals derived from flax and coco.

These compounds are classified in the CTFA dictionary, 5th edition, 1993, under the names disodium cocoamphodiacetate, disodium lauroamphodiacetate, disodium caprylamphodiacetate, disodium capryloamphodiacetate, disodium cocoamphodipropionate, disodium lauroamphodipropionate, disodium caprylamphodipropionate, disodium capryloamphodipropionate, lauroamphodipropionic acid, and cocoamphodipropionic acid.

By way of non-limiting example, mention may be made of the sodium cocoamphodiacetate sold under the trade name MIRANOL C2M concentrated by the company Rhodia Chimie.

(iv) The cationic surfactants may be chosen from:

A) the quaternary ammonium salts of formula (XIX):

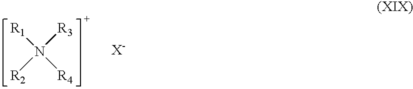

wherein X is an anion chosen from thalides, such aschloride, bromide and iodide; (C₂-C₆)alkyl sulfates, for example methyl sulfate, phosphates, alkyl, and alkylaryl sulfonates, and anions derived from organic acid, such as acetate or lactate, and

a) the radicals R₁ to R₃, which may be identical or different, are chosen from C₁-C₄ linear and branched aliphatic radicals and aromatic radicals, such as aryl or alkylaryl. The aliphatic radicals can comprise heteroatoms such as oxygen, nitrogen, sulfur, and halogens. The aliphatic radicals are chosen, for example from alkyl, alkoxy, and alkylamide radicals,

R₄ is chosen from a C₁₆-C₃₀ linear and branched alkyl radical.

The cationic surfactant is chosen, for instance, from behenyltrimethylammonium, cetyltrimethylammonium, and cetearyltrimethylammonium salts, for example, chloride.

b) the radicals R₁ and R₂, which may be identical or different, are chosen from C₁-C₄ linear and branched aliphatic radicals and aromatic radicals, such as aryl or alkylaryl. The aliphatic radicals can comprise heteroatoms such as oxygen, nitrogen, sulfur, and halogens. The aliphatic radicals are chosen, for example, from alkyl, alkoxy, alkylamide, and hydroxyalkyl radicals comprising from about 1 to 4 carbon atoms;

R₃ and R₄, which may be identical or different, are chosen from C₁₂-C₃₀ linear and branched alkyl radicals, wherein the radicals comprise at least one ester or amide function.

R₃ and R₄ are chosen, for instance, from (C₁₂-C₂₂)alkylamido(C₂-C₆)alkyl and (C₁₂-C₂₂)alkylacetate radicals.

The cationic surfactant is, in at least one embodiment, a stearamidopropyldimethyl(myristyl acetate)ammonium salt, such as chloride.

B)—the quaternary ammonium salts of imidazolinium, for example, of formula (XX) below:

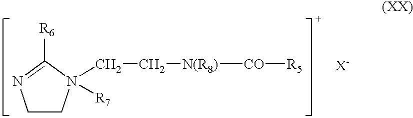

wherein R₅ is chosen from a C₈-C₃₀ alkenyl and alkyl radical, for example fatty acid derivatives of tallow, R₆ is chosen from a hydrogen atom, a C₁-C₄ alkyl radical, and a C₈-C₃₀ alkenyl and alkyl radical, R₇ is chosen from a C₁-C₄ alkyl radical, R₈ is chosen from a hydrogen atom and a C₁-C₄ alkyl radical, such as a hydrogen atom, and X is an anion chosen from halides, phosphates, acetates, lactates, alkyl sulfates, alkyl sulfonates, and alkylaryl sulfonates. In at least one embodiment, R₅ and R₆ are chosen from a mixture of alkenyl and alkyl radicals comprising from 12 to 21 carbon atoms, such as, fatty acid derivatives of tallow or palm oil, R₇ is methyl, and R₈ is a hydrogen atom. Such a product is, for example, Quaternium-27 (CTFA 1997) or Quaternium-83 (CTFA 1997), which are sold under the names REWOQUAT W75, W90, W75PG, and W75HPG by the company Witco, and VARISOFT W575 PG by the company Degussa,

C)— the diquaternary ammonium salts of formula (XXI):

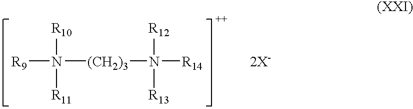

wherein R₉ is chosen from a C₁₆-C₃₀ aliphatic radical, R₁₀, R₁₁, R₁₂, R₁₃, and R₁₄, which may be identical or different, are chosen from hydrogen atoms and C₁-C₄ alkyl radicals, and X is an anion chosen from halides, acetates, phosphates, nitrates, and methyl sulfates. Such diquaternary ammonium salts comprise, for example, propane tallow diammonium dichloride;

D)—the quaternary ammonium salts comprising at least one ester function of formula (XXII):

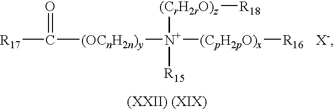

wherein:

R₁₅ is chosen from a C₁-C₆ alkyl radical and a C₁-C₆ hydroxyalkyl and dihydroxyalkyl radical;

R₁₆ is chosen from:
a radical

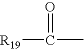

R₂₀, wherein R₂₀ is chosen from linear and branched, saturated and unsaturated C₁-C₂₂ hydrocarbon radicals, and
a hydrogen atom,

R₁₈ is chosen from:
a radical

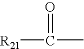

R₂₂, wherein R₂₂ is chosen from linear and branched, saturated and unsaturated C₁-C₆ hydrocarbon radicals, and
a hydrogen atom,

R₁₇, R₁₉, and R₂₁, which may be identical or different, are chosen from linear and branched, saturated and unsaturated C₇-C₂₁ hydrocarbon radicals;

n, p, and r, which may be identical or different, are integers ranging from 2 to 6;

y is an integer ranging from 1 to 10;

x and z, which may be identical or different, are integers ranging from 0 to 10;

X⁻ is a simple or complex, organic or inorganic anion;

with the proviso that the sum x+y+z is from 1 to 15, that when x is 0, then R₁ is R₂₀, and that when z is 0, then R₁₈ is R₂₂.

Useful ammonium salts that may be mentioned herein, include those of formula (XXII) wherein:

R₁₅ is a methyl or ethyl radical;

x and y are 1;

z is 0 or 1;

n, p, and r are 2;

R₁₆ is chosen from:
a radical

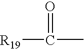

methyl, ethyl, and C₁₄-C₂₂ hydrocarbon radicals, and
a hydrogen atom;

R₁₇, R₁₉, and R₂₁, which may be identical or different, are chosen from linear and branched, saturated and unsaturated C₇-C₂₁ hydrocarbon radicals;

R₁₈ is chosen from:
a radical

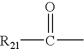

and a hydrogen atom.

Such compounds are sold, for example, under the names DEHQUART by the company Cognis, STEPANQUAT by the company Stepan, NOXAMIUM by the company Ceca, and REWOQUAT WE 18 by the company Rewo-Witco.

Non-limiting mention may be made among the quaternary ammonium salts of behenyltrimethylammonium chloride and stearamidopropylmethyl(myristyl acetate)ammonium chloride, sold, for instance under the name CERAPHYL 70 by the company Van Dyk, and QUATERNIUM-27 or QUATERNIUM-83 sold by the company Witco.

In the compositions according to the present disclosure, mixtures of surfactants such as mixtures of anionic surfactants, mixtures of anionic surfactants and of amphoteric, cationic or nonionic surfactants, and mixtures of cationic surfactants with nonionic or amphoteric surfactants may be used. In at least one embodiment, a mixture comprising at least one anionic surfactant and at least one amphoteric surfactant may be used.

The composition of the present disclosure may also comprise at least one additive chosen from thickeners, fragrances, nacreous agents, preserving agents, silicone and non-silicone sunscreens, anionic and nonionic polymers, proteins, protein hydrolysates, 18-methyleicosanoic acid, hydroxy acids, vitamins, provitamins such as panthenol, silicones, anti-dandruff or anti-seborrhoeic agents, hair-loss counteractants and/or hair restorers, electrolytes, proteins, protein hydrolysates, fluoro or perfluoro oils, natural or synthetic waxes, ceramide type compounds, fatty amines, fatty acids and derivatives thereof, fatty alcohols and derivatives thereof, and also mixtures of these various compounds, and any other additive conventionally used in cosmetics that does not affect the properties of the compositions disclosed herein.

The compositions in accordance with the present disclosure may also comprise from 0% to 5% of nacreous or opacifying agents that are well known in the prior art, such as sodium and magnesium palmitate, sodium and magnesium stearate and hydroxystearate, fatty-chain acyl derivatives such as ethylene glycol and polyethylene glycol monostearates and distearates, fatty-chain ethers, such as C₁₀-C₃₀ fatty-chain ethers, for example, distearyl ether or 1-(hexadecyloxy)-2-octadecanol, and fatty alcohols, such as stearyl alcohol, cetyl alcohol, behenyl alcohol, and mixtures thereof.

These additives are optionally present in the compositions disclosed herein in an amount ranging from 0.001% to 20% by weight, relative to the total weight of the composition. The precise amount of each additive is readily determined by a person skilled in the art on the basis of its nature and its function.

According to the present disclosure, the physiologically and cosmetically acceptable medium may consist solely of water or of a mixture of water and a cosmetically acceptable solvent such as a C₁-C₄ lower alcohol, for instance, ethanol, isopropanol, tert-butanol, and n-butanol; alkylene glycols, for instance propylene glycol, and glycol ethers.

In one embodiment, the composition comprises water in an amount ranging from 50% to 95% by weight, relative to the total weight of the composition, such as from 60 to 90% by weight.

The compositions disclosed herein have a final pH generally between 3 and 10. According to one embodiment, this pH is between 4 and 8 such as between 4 and 6. Adjusting the pH to the desired value may be performed conventionally by adding a base (organic or mineral base) to the composition, for example aqueous ammonia or a primary, secondary or tertiary (poly)amine, for instance monoethanolamine, diethanolamine, triethanolamine, isopropanolamine, and 1,3-propanediamine, or by adding a mineral or organic acid, such as a carboxylic acid, for example, citric acid.

When the composition is in the form of a hair conditioner optionally to be rinsed out, it may further comprise at least one cationic surfactant, the concentration ranging from 0.1% to 10% by weight, such as from 0.5% to 5% by weight, relative to the total weight of the composition.

The compositions disclosed herein may also be used, for instance, for washing or treating keratin materials such as the hair, the skin, the eyelashes, the eyebrows, the nails, the lips, or the scalp, and in at least one embodiment, the hair. In at least one embodiment, the compositions according to the present disclosure may also be detergent compositions such as shampoos, shower gels, and bubble baths and may further comprise at least one detergent surfactant chosen, for instance, from anionic, amphoteric, nonionic surfactants, and mixtures thereof.

When the compositions disclosed herein are in the form of detergent compositions such as shampoos, at least one anionic surfactant or mixtures of at least one anionic surfactant and of at least one amphoteric surfactant or of at least one nonionic surfactant may be used.

According to another embodiment, a mixture comprising at least one anionic surfactant and at least one amphoteric surfactant is used.

Use may be made, for example, of an anionic surfactant chosen from sodium, triethanolamine and ammonium (C₁₂-C₁₄)alkyl sulfates, sodium, triethanolamine and ammonium (C₁₂-C₁₄)alkyl ether sulfates oxyethylenated comprising 2.2 mol of ethylene oxide, sodium cocoyl isethionate and sodium α-(C₁₄-C₁₆)olefin sulfonate, and mixtures thereof with:

either an amphoteric surfactant such as the amine derivatives known as disodium cocoamphodipropionate or sodium cocoamphopropionate sold, for example, by the company Rhodia Chimie under the trade name MIRANOL C2M Conc. as an aqueous solution comprising 38% active material, or under the name MIRANOL C32;

or an amphoteric surfactant of zwitterionic type, such as alkylbetaines or alkylamidobetaines and, for example, the cocobetaine sold under the name DEHYTON AB 30 as an aqueous solution comprising 32% AM by the company Cognis.

Suitable non-limiting examples of the at least one anionic surfactant include, but are not limited to, sodium, triethanolamine and ammonium (C₁₂-C₁₄)alkyl sulfates, sodium, triethanolamine and ammonium (C₁₂-C₁₄)alkyl ether sulfates oxyethylenated comprising 2.2 mol of ethylene oxide, sodium cocoyl isethionate and sodium x-(C₁₄-C₁₆)olefin sulfonate, and mixtures thereof with:

either an amphoteric surfactant such as the amine derivatives known as disodium cocoamphodipropionate or sodium cocoamphopropionate sold, for instance, by the company Rhodia Chimie under the trade name MIRANOL C2M Conc. as an aqueous solution comprising 38% active material, or under the name MIRANOL C32;

or an amphoteric surfactant of zwitterionic type, such as alkylbetaines, such as the cocobetaine sold under the name DEHYTON AB 30 as an aqueous solution comprising 32% AM by the company Cognis, and the cocoamidopropylbetaine sold, for instance by Goldschmidt under the name TEGOBETAINE F50.

The quantity and quality of the washing base is chosen in order to give the final composition satisfactory foaming power and/or detergent power.

These detergent compositions may be, for example, foaming compositions and the foaming power of the compositions disclosed herein, characterized by a foam height, may be greater, for instance, than 75 mm, such as greater than 100 mm, measured according to the modified Ross-Miles method (NF T 73-404/IS696).

The modifications to the method are the following:

The measurement is performed at a temperature of 22° C. with osmosed water. The concentration of the solution is 2 g/l. The height of the drop is 1 m. The amount of composition that is dropped is 200 ml. These 200 ml of composition fall into a measuring cylinder 50 mm in diameter and containing 50 ml of the test composition. The measurement is taken 5 minutes after stopping the flow of the composition.

Thus, according to the present disclosure, in these detergent compositions, the surfactants may be present in an amount ranging from 3% to 50% by weight, for example from 6% to 35% by weight, and further for example from 8% to 25% by weight, relative to the total weight of the final composition.

Another aspect of the present disclosure is a process for treating keratin materials such as the skin or the hair, comprising applying to the keratin materials a cosmetic composition as disclosed herein, and then optionally rinsing with water after an optional leave-in time.

Thus, the process disclosed herein allows holding of the hairstyle, and treatment, care and washing of, or makeup removal from the skin, the hair or any other keratin material.

The compositions disclosed herein may also be in the form of permanent-waving, hair-relaxing, dyeing, or bleaching compositions, or in the form of rinse-out compositions to be applied before or after dyeing, bleaching, permanent-waving or relaxing the hair or between the two steps of a permanent-waving or hair-relaxing operation.

The compositions of the present disclosure may also be in the form of washing compositions for the skin, such as in the form of bath or shower solutions or gels, or makeup-removing products.

The compositions according to the present disclosure may also be in the form of aqueous or aqueous-alcoholic lotions for skincare and/or haircare.

The cosmetic compositions according to the invention may further be in the form of a gel, a milk, a cream, an emulsion, a thickened lotion, or a mousse and may be used for the skin, the nails, the eyelashes, the lips and, such as the hair.

The compositions may be conditioned in various forms, such as vaporizers, pump-dispenser bottles, and aerosol containers to allow an application of the composition in vaporized form or in the form of a mousse. Such conditioning forms are indicated, for example, when it is desired to obtain a spray, a lacquer or a mousse for treating keratin materials such as the hair.

As disclosed herein, the percentages stated are by weight.

Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the disclosure are approximations, unless otherwise indicated the numerical values set forth in the specific examples are reported as precisely as possible. Any numerical value, however, inherently contains certain errors necessarily resulting from the standard deviation found in their respective testing measurements.

The examples that follow are intended to illustrate the present disclosure without, however, being limiting in nature.

In the examples, AM means active material.

### EXAMPLE 1

A shampoo in accordance with the present disclosure, having the following composition, was prepared:

| Composition | 1 | |
|---|---|---|
| Sodium lauryl ether sulfate comprising 2.2 mol of | 10.5 | g AM |
| ethylene oxide, as an aqueous solution comprising | | |
| 70% AM | | |
| Cocoylamidopropylbetaine as an aqueous solution | 1.5 | g AM |
| comprising 30% AM (TEGOBETAINE F 50 from | | |
| Goldschmidt) | | |
| Copolymer of acrylic or methacrylic acid esters, of | 1.2 | g AM |
| C1-4 dialkylamino C1-6 alkyl methacrylate, of | | |
| PEG/PPG-30/5 allyl ether, of C10-30 PEG 20-25 alkyl | | |
| ether methacrylate and of C2-6 hydroxyalkyl methacrylate | | |
| crosslinked with ethylene glycol | | |
| dimethacrylate, as a 20% emulsion in water | | |
| (CARBOPOL Aqua CC Polymer from Noveon) | | |
| Olive oil | 1 | g |
| Preserving agents, dye | qs | |
| Citric acid or sodium hydroxide qs | pH 5.5 | |
| Demineralized water qs | 100 | g |

This thickened shampoo had a pleasant texture and was stable over time, and was easy to apply and to rinse out.

It gave the hair smoothness, softness, and sheen.

### EXAMPLES 2 and 3

The shampoos in accordance with the present disclosure, having the following composition, were prepared:

| Composition | 2 | | 3 | |
|---|---|---|---|---|
| Sodium lauryl ether sulfate comprising 2.2 mol of | 10.5 | g AM | 10.5 | g AM |
| ethylene oxide, as an aqueous solution comprising 70% AM | | | | |
| Cocoylamidopropylbetaine as an aqueous solution | 1.5 | g AM | 1.5 | g AM |
| comprising 30% AM (TEGOBETAINE F 50 from | | | | |
| Goldschmidt) | | | | |
| Copolymer of acrylic or methacrylic acid esters, of | 1 | g AM | 2 | g AM |
| C1-4 dialkylamino C1-6 alkyl methacrylate, of | | | | |
| PEG/PPG-30/5 allyl ether, of C10-30 PEG 20-25 | | | | |
| alkyl ether methacrylate and of C2-6 hydroxyalkyl | | | | |
| methacrylate crosslinked with ethylene glycol | | | | |
| dimethacrylate, as a 20% emulsion in water | | | | |
| (CARBOPOL Aqua CC from Noveon) | | | | |
| Olive oil | 1 | g | 1 | g |
| Hydroxypropyl guar trimethylammonium chloride | | | 0.1 | g |
| (JAGUAR EXCELL from Rhodia) | | | | |
| Cationic cellulose ether (QUATRISOFT LM 200 from | 0.2 | g | | |
| Amerchol) | | | | |
| Preserving agents, dye | qs | | qs | |
| Citric acid or sodium hydroxide qs qs | pH 5.5 | | pH 5.5 | |
| Deionized water qs | 100 | g | 100 | g |

The compositions had a thick gel texture and were stable. The working qualities of the products were good (the foam was rich and creamy), and rinsing was quick and easy.

Wet hair treated with this shampoo was crisp but supple, and disentangled well; dry hair had body and sheen.

### EXAMPLE 4

The shampoo in accordance with the present disclosure, having the following composition, was prepared:

| Composition | 4 | |
|---|---|---|
| Sodium lauryl ether sulfate comprising 2.2 mol of ethylene | 10.5 | g AM |
| oxide, as an aqueous solution comprising 70% AM | | |
| Cocoylamidopropylbetaine as an aqueous solution | 1.5 | g AM |
| comprising 30% AM (TEGOBETAINE F 50 from | | |
| Goldschmidt) | | |
| Copolymer of acrylic or methacrylic acid esters, of C1-4 | 2 | g AM |
| dialkylamino C1-6 alkyl methacrylate, of PEG/PPG-30/5 | | |
| allyl ether, of C10-30 PEG 20-25 alkyl ether methacrylate | | |
| and of C2-6 hydroxyalkyl methacrylate crosslinked with | | |
| ethylene glycol dimethacrylate, as a 20% emulsion in | | |
| water (CARBOPOL AQUAC from Noveon) | | |
| Olive oil | 5 | g |
| Hydroxypropyl guar trimethylammonium chloride | 0.2 | g |
| (JAGUAR EXCELL from Rhodia) | | |
| Preserving agents, dye | qs | |
| Citric acid or sodium hydroxide qs | pH 5.5 | |
| Deionized water qs | 100 | g |

The composition had a thick gel texture and was stable. The working qualities of the product were good (the foam was rich and creamy), and rinsing was quick and easy.

Wet hair treated with this shampoo was crisp but supple, and disentangled well; dry hair had body and sheen.

### EXAMPLES 5 and 6

The shampoos in accordance with the present disclosure, having the following composition, were prepared:

| Composition | 5 | | 6 | |
|---|---|---|---|---|
| Sodium lauryl ether sulfate comprising 2.2 mol of | 10.5 | g AM | 10.5 | g AM |
| ethylene oxide, as an aqueous solution comprising 70% AM | | | | |
| Cocoylamidopropylbetaine as an aqueous solution | 1.5 | g AM | 1.5 | g AM |
| comprising 30% AM (TEGOBETAINE F 50 from | | | | |
| Goldschmidt) | | | | |
| Copolymer of acrylic or methacrylic acid esters, of | 1 | g AM | 1 | g AM |
| C1-4 dialkylamino C1-6 alkyl methacrylate, of | | | | |
| PEG/PPG-30/5 allyl ether, of C10-30 PEG 20-25 | | | | |
| alkyl ether methacrylate and of C2-6 hydroxyalkyl | | | | |
| methacrylate crosslinked with ethylene glycol | | | | |
| dimethacrylate, as a 20% emulsion in water | | | | |
| (CARBOPOL AQUA CC from Noveon) | | | | |
| Olive oil | 5 | g | 1 | g |
| Cationic cellulose ether (QUATRISOFT LM 200 from | 0.4 | g | 0.2 | g |
| Amerchol) | | | | |
| Preserving agents, dye | qs | | qs | |
| Citric acid or sodium hydroxide qs qs | pH 5.5 | | pH 5.5 | |
| Deionized water qs | 100 | g | 100 | g |

### EXAMPLE 7

A hair conditioner having the following composition was prepared:

| Avocado oil | 5.25 | g |
|---|---|---|
| Liquid jojoba wax | 5.25 | g |
| Cyclopentadimethylsiloxane | 3.5 | g |
| Dipropylene glycol | 10 | g |
| Glycerol | 5 | g |
| Polydimethyl/methyl aminoethyl aminopropyl siloxane as | 1.2 | g AM |
| a nonionic microemulsion comprising 20% AM | | |
| Behenyltrimethylammonium chloride as a solution in a | 0.5 | g AM |
| water/isopropanol mixture | | |
| Oxyethylenated (20 EO) sorbitan monolaurate | 0.5 | g |
| Polyethylene glycol monoisostearate (8 EO) | 2 | g |
| Lactic acid qs | pH 4 | |
| Fragrance, preserving agents | qs | |
| Deionized water qs | 100 | g |

This hair conditioner had a pleasant thickened texture and was stable over time. After application to the hair, this composition was easy to rinse out, and gave the hair disentangling, smoothness, softness, and sheen.

What is claimed is:

1. A cosmetic composition, comprising, in a cosmetically acceptable medium:
(i) at least one cationic polymer, produced by polymerization of a monomer mixture comprising:
a) at least one vinyl monomer substituted with at least one amino group,
b) at least one hydrophobic nonionic vinyl monomer, chosen from formulae (I) and (II):
CH₂═C(X)Z, (I)
CH₂═CH—OC(O)R; (II)
wherein:
X is chosen from a hydrogen atom and a methyl group;
Z is chosen from the groups —C(O)OR¹, —C(O)NH₂, —C(O)NHR¹, —C(O)N(R¹)₂, —C₆H₅, —C₆H₄R¹, —C₆H₄OR¹, —C₆H₄Cl, —CN, —NHC(O)CH₃, —NHC(O)H, N-(2-pyrrolidonyl), N-caprolactamyl, —C(O)NHC(CH₃)₃, —C(O)NHCH₂CH₂—NH—CH₂CH₂-urea, —Si(R)₃, —C(O)O(CH₂)ₓSi(R)₃, —C(O)NH(CH₂)ₓSi(R)₃, and —(CH₂)ₓSi(R)₃;
x is an integer ranging from 1 to 6;
each R is independently chosen from a C₁-C₃₀ alkyl group;
each R¹ is independently chosen from a C₁-C₃₀ alkyl group, a C₂-C₃₀ hydroxyalkyl group, and a C₁-C₃₀ haloalkyl group, and
c) at least one associative vinyl monomer, and
e) at least one hydroxylated nonionic vinyl monomer,
(ii) at least one saturated or unsaturated C12-C30 fatty acid triglyceride, the content of C18:2 acids of the triglyceride being less than 50% by weight relative to the total weight of the fatty acids of the triglyceride, and
(iii) at least one non-silicone cationic polymer other than the at least one cationic polymer (i).

2. The composition according to claim 1, wherein the at least one vinyl monomer substituted with at least one amino group a) is chosen from:
mono(C₁-C₄)alkylamino(C₁-C₈)alkyl (meth)acrylates,
di(C₁-C₄)alkylamino(C₁-C₈)alkyl (meth)acrylates,
mono(C₁-C₄)alkylamino(C₁-C₈)alkyl(meth)acrylamides,
di(C₁-C₄)alkylamino(C₁-C₈)alkyl(meth)acrylamides,
heterocyclic (meth)acrylamides comprising a nitrogen atom,
heterocyclic (meth)acrylates comprising a nitrogen atom,
and mixtures thereof.

3. The composition according to claim 2, wherein the at least one vinyl monomer substituted with at least one amino group a) is chosen from:
mono- and di(C₁-C₄ alkyl)amino(C₁-C₄ alkyl) (meth)acrylates;
mono- and di(C₁-C₄ alkyl)amino(C₁-C₄ alkyl)(meth)acrylamides;
(meth)acrylamides and (meth)acrylates with a heterocyclic group comprising a nitrogen atom; and
nitrogenous heterocycles comprising at least one vinyl group.

4. The composition according to claim 1, wherein the at least one vinyl monomer substituted with at least one amino group a) is present in an amount ranging from 10% to 70% by weight, relative to the total weight of the monomer mixture.

5. The composition according to claim 1, wherein the at least one hydrophobic nonionic vinyl monomer b) is chosen from C₁-C₃₀ alkyl (meth)acrylates, (C₁-C₃₀ alkyl)(meth)acrylamides, styrene, substituted styrenes, vinyl esters, unsaturated nitriles, and unsaturated silanes.

6. The composition according to claim 1, wherein the at least one hydrophobic nonionic vinyl monomer b) is present in an amount ranging from 20% to 80% by weight, relative to the total weight of the monomer mixture.

7. The composition according to claim 1, wherein the at least one associative vinyl monomer c) is chosen from compounds of formula (III): 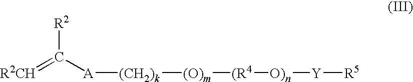
wherein:
each R² is independently chosen from a hydrogen atom, a methyl group, —C(O)OH, and —C(O)OR³;
R³ is a C₁-C₃₀ alkyl;
A is chosen from —CH₂C(O)O—, —C(O)O—, —O—, CH₂O, —NHC(O)NH—, —C(O)NH—, —Ar—(CE₂)_{z}—NHC(O)O—, —Ar—(CE₂)_{z}-NHC(O)NH—, and —CH₂CH₂—NHC(O)—;
Ar is a divalent aryl group;
E is chosen from a hydrogen atom and a methyl group;
z is an integer ranging from 0 to 1;
k is an integer ranging from 0 to 30;
m is an integer ranging from 0 to 1, with the provisos that when k is 0, then m is 0, and when k is an integer ranging from 1 to 30, then m is 1;
(R⁴—O)ₙ is a polyoxyalkylene, which is a homopolymer, a random copolymer, or a block copolymer, with C₂-C₄ oxyalkylene units;
R⁴ is chosen from C₂H₄, C₃H₆, C₄H₈, and mixtures thereof;
n is an integer ranging from 5 to 250;
Y is chosen from —R⁴⁰—, —R⁴NH—, —C(O)—, —C(O)NH—, R⁴NHC(O)NH—, and —C(O)NHC(O)—;
R⁵ is chosen from substituted and unsubstituted alkyls chosen from linear C₈-C₄₀ alkyl groups, branched C₈-C₄₀ alkyl groups, C₈-C₄₀ alicyclic groups, phenyls substituted with a C₂-C₄₀ alkyl group, C₂-C₄₀ alkyl groups substituted with an aryl group, and C₈-C₈₀ complex esters,
wherein the alkyl group R⁵ optionally comprises at least one substituent chosen from hydroxyl, alkoxy, and halo groups.

8. The composition according to claim 7, wherein the at least one associative vinyl monomer c) is chosen from polyethoxylated cetyl (meth)acrylates, polyethoxylated cetearyl (meth)acrylates, polyethoxylated stearyl (meth)acrylates, polyethoxylated arachidyl (meth)acrylates, polyethoxylated behenyl (meth)acrylates, polyethoxylated lauryl (meth)acrylates, polyethoxylated cerotyl (meth)acrylates, polyethoxylated montanyl (meth)acrylates, polyethoxylated melissyl (meth)acrylates, polyethoxylated lacceryl (meth)acrylates, polyethoxylated 2,4,6-tris(1′-phenylethyl)phenyl (meth)acrylates, polyethoxylated hydrogenated castor oil (meth)acrylates, polyethoxylated canola (meth)acrylates, polyethoxylated cholesteryl (meth)acrylates, and mixtures thereof, wherein the polyethoxylated portion of the monomer comprises from 5 to 100 ethylene oxide units.

9. The composition according to claim 1, wherein the at least one associative vinyl monomer c) is present in an amount ranging from 0.001% to 25% by weight, relative to the weight of the monomer mixture.

10. The composition according to claim 1, wherein the monomer mixture further comprises at least one semi-hydrophobic vinyl surfactant monomer d) chosen from compounds of formula (IV) and (V): 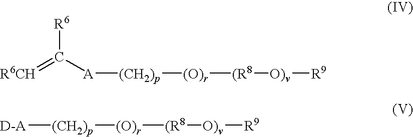
wherein:
each R⁶ is independently chosen from a hydrogen atom, a C₁-C₃₀ alkyl, —C(O)OH, and C(O)OR⁷;
R⁷ is a C₁-C₃₀ alkyl;
A is chosen from —CH₂C(O)O—, —C(O)O—, —O—, —CH₂O, —NHC(O)NH—, —C(O)NH—, —Ar—(CE₂)_{z}—NHC(O)O—, —Ar—(CE₂)_{z}-NHC(O)NH—, and —CH₂CH₂NHC(O)—;
Ar is a divalent aryl group;
E is chosen from a hydrogen atom and a methyl group;
z is an integer ranging from 0 to 1;
p is an integer ranging from 0 to 30;
r is an integer ranging from 0 to 1, with the provisos that when p is 0, then r is 0, and when
p is an integer ranging from 1 to 30, then r is 1,
(R₈—O)ᵥ is a polyoxyalkylene which is a homopolymer, a random copolymer or a block copolymer with C₂-C₄ oxyalkylene units, wherein R⁸ is chosen from C₂H₄, C₃H₆, C₄H₈, and mixtures thereof and v is an integer ranging from 5 to 250;
R⁹ is chosen from a hydrogen atom and a C₁-C₄ alkyl; and
D is chosen from C₈-C₃₀ alkenyl groups optionally substituted with a carboxyl group.

11. The composition according to claim 10, wherein the monomer mixture comprises at least one semi-hydrophobic vinyl surfactant monomer d) chosen from one of the following formulae:
CH₂═CH—O(CH₂)ₐO(C₃H₆O)_{b}(C₂H₄O)_{c}H, and
CH₂═CHCH₂O(C₃H₆O)_{d}(C₂H₄₀)ₑH;
wherein:
a is an integer ranging from 2 to 4;
b is an integer ranging from 1 to 10;
c is an integer ranging from 5 to 50;
d is an integer ranging from 1 to 10; and
e is an integer ranging from 5 to 50.

12. The composition according to claim 10, wherein the at least one semi-hydrophobic vinyl surfactant monomer d) is present in an amount ranging from 0% to 25% by weight, relative to the total weight of the monomer mixture.

13. The composition according to claim 1, wherein the at least one hydroxylated nonionic vinyl monomer e) is chosen from C₁-C₆ hydroxyalkyl (meth)acrylates, (C₁-C₄ hydroxyalkyl)(meth)acrylamides, and mixtures thereof.

14. The composition according to claim 13, wherein the at least one hydroxylated nonionic vinyl monomer e) is 2-hydroxyethyl methacrylate.

15. The composition according to claim 1, wherein the at least one hydroxylated nonionic vinyl monomer e) is present in an amount ranging from 0% to 10% by weight, relative to the total weight of the monomer mixture.

16. The composition according to claim 15, wherein the monomer mixture comprises, relative to the total weight of the monomer mixture:
a) from 20% to 60% by weight of at least one vinyl monomer substituted with at least one amino group a),
b) from 20% to 70% by weight of at least one hydrophobic nonionic vinyl monomer b),
c) from 0.01% to 15% by weight of at least one associative vinyl monomer c),
d) from 0.1% to 10% by weight of at least one semi-hydrophobic vinyl surfactant monomer d),
e) from 0.01% to 10% by weight of at least one hydroxylated nonionic vinyl monomer e),
f) from 0.001% to 5% by weight of at least one crosslinking monomer,
g) from 0.001% to 10% by weight of at least one chain-transfer agent, and
h) from 0 to 2% by weight of at least one polymeric stabilizer.

17. The composition according to claim 1, wherein the monomer mixture comprises:
a di(C₁-C₄ alkyl)amino(C₁-C₆ alkyl)methacrylate,
at least one C₁-C₃₀ alkyl ester of (meth)acrylic acid,
a C₁₀-C₃₀ alkyl methacrylate polyethoxylated comprising 20 to 30 mol of ethylene oxide,
a 30/5 polyethylene glycol/polypropylene glycol allyl ether,
a hydroxy(C₂-C₆ alkyl)methacrylate, and
an ethylene glycol dimethacrylate.

18. The composition according to claim 1, wherein the at least one cationic polymer (i) is present in an amount ranging from 0.01% to 10% by weight, relative to the total weight of the composition.

19. The composition according to claim 1, wherein the at least one triglyceride (ii) is chosen from triglycerides of sweet almond oil, avocado oil, castor oil, olive oil, sesameseed oil, groundnut oil, rapeseed oil, canola oil, macadamia nut oil, coconut oil, hazelnut oil, cashew nut oil, yellow flax (camelina) oil, shea buffer, palm oil, apricot kernel oils argan oil, beauty-leaf oil, and grapeseed oil, and mixtures thereof.

20. The composition according to claim 19, wherein the at least one triglyceride (ii) is chosen from triglycerides of avocado oil, olive oil, apricot kernel oil, coconut oil, palm oil, and argan oil, and mixtures thereof.

21. The composition according to claim 1, wherein the at least one triglyceride (ii) is present in a amount ranging from 0.001% and 20% by weight, relative to the total weight of the composition.

22. The composition according to claim 1, wherein the at least one cationic polymer (iii) is chosen from cationic cyclopolymers, cationic polysaccharides, quaternary polymers of vinylpyrrolidone and of vinylimidazole, and crosslinked homopolymers or copolymers of methacryloyloxy(C₁-C₄)alkyltri(C₁-C₄)alkylammonium salts, and mixtures thereof.

23. The composition according to claim 22, wherein the cyclopolymer is chosen from diallyldimethylammonium chloride homopolymers and copolymers of diallyldimethylammonium chloride and of acrylamide.

24. The composition according to claim 22, wherein the at least one cationic polysaccharide is chosen from guar gums modified with a 2,3-epoxypropyl trimethylammonium salt and hydroxyethylcelluloses that have reacted with an epoxide substituted with a trimethylammonium group.

25. The composition according to claim 1, wherein the at least one cationic polymer (iii) is present in an amount ranging from 0.001% and 20% by weight, relative to the total weight of the composition.

26. The composition according to claim 1, further comprising at least one surfactant chosen from anionic, nonionic, amphoteric, cationic surfactants, and mixtures thereof.

27. The composition according to claim 26, wherein the at least one surfactant is present in an amount ranging from 0.01% and 50% by weight, relative to the total weight of the composition.

28. The composition according to claim 1, wherein the composition comprises at least one additive chosen from thickeners, antidandruff agents or anti-seborrhoeic agents, fragrances, nacreous agents, hydroxy acids, electrolytes, preserving agents, silicone and non-silicone sunscreens, vitamins, provitamins, anionic and nonionic polymers, proteins, protein hydrolysates, 18-methyleicosanoic acid, fluoro and perfluoro oils, silicones, natural and synthetic waxes, compounds of ceramide type, fatty amines, fatty acids and derivatives thereof, fatty alcohols and derivatives thereof, and also mixtures of these various compounds.

29. The composition according to claim 1, wherein the composition is in the form of a rinse-out or leave-in hair-conditioning composition.

30. The composition according to claim 1, wherein the composition is in the form of a foaming detergent composition chosen from shampoos, shower gels, makeup-removing products, and bubble baths.

31. The composition according to claim 1, wherein the composition is in the form of a permanent-waving, hair-relaxing, dyeing, or bleaching composition, or in the form of a composition to be applied before or after dyeing, bleaching, permanent-waving, or relaxing the hair or between the two steps of a permanent-waving or hair-relaxing operation.

32. A process for treating keratin materials, comprising applying to the keratin materials a cosmetic composition comprising, in a cosmetically acceptable medium:
(i) at least one cationic polymer, produced by polymerization of a monomer mixture comprising:
a) at least one vinyl monomer substituted with at least one amino group,
b) at least one hydrophobic nonionic vinyl monomer, chosen from formulae (I) and (II):
CH₂═C(X)Z, (I)
CH₂═CH—OC(O)R; (II)
wherein:
X is chosen from a hydrogen atom and a methyl group;
Z is chosen from the groups —C(O)OR¹, —C(O)NH₂, —C(O)NHR¹, —C(O)N(R¹)₂, —C₆H₅, —C₆H₄R¹, —C₆H₄OR¹, —C₆H₄Cl, —CN, —NHC(O)CH₃, —NHC(O)H, N-(2-pyrrolidonyl), N-caprolactamyl, —C(O)NHC(CH₃)₃, —C(O)NHCH₂CH₂—NH—CH₂CH₂-urea, —Si(R)₃, —C(O)O(CH₂)ₓSi(R)₃, —C(O)NH(CH₂)ₓSi(R)₃ and —(CH₂)ₓSi(R)₃;
x is an integer ranging from 1 to 6;
each R is independently chosen from a C₁-C₃₀ alkyl group;
each R¹ is independently chosen from a C₁-C₃₀ alkyl group, a C₂-C₃₀ hydroxyalkyl group, and a C₁-C₃₀ haloalkyl group, and
c) at least one associative vinyl monomer, and
e) at least one hydroxylated nonionic vinyl monomer,
(ii) at least one saturated or unsaturated C₁₂-C₃₀ fatty acid triglyceride, the content of C18:2 acids of the triglyceride being less than 50% by weight relative to the total weight of the fatty acids of the triglyceride, and
(iii) at least one non-silicone cationic polymer other than the at least one cationic polymer (i),
optionally followed by rinsing with water, after an optional leave-in time.
